(19) United States
(12) Patent Application Publication
(10) Pub. No.: US 2024/0383910 A1
(43) Pub. Date: Nov. 21, 2024

(54) SMALL MOLECULE BTK DEGRADERS AND METHODS OF USE THEREOF

(71) Applicant: Baylor College of Medicine, Houston, TX (US)

(72) Inventors: Wen-Hao Guo, Houston, TX (US); Xin Yu, Houston, TX (US); Ran Cheng, Houston, TX (US); Jin Wang, Houston, TX (US)

(73) Assignee: Baylor College of Medicine, Houston, TX (US)

(21) Appl. No.: 18/664,964

(22) Filed: May 15, 2024

Related U.S. Application Data
(60) Provisional application No. 63/502,209, filed on May 15, 2023.

(51) Int. Cl. C07D 491/048; A61K 31/454; A61K 31/496; A61K 31/506; A61K 31/519; A61K 45/06; C07D 401/04; C07D 401/14; C07D 403/12

(52) U.S. Cl. CPC C07D 491/048; A61K 31/454; A61K 31/496; A61K 31/506; A61K 31/519; A61K 45/06; C07D 401/04; C07D 401/14; C07D 403/12

(57) ABSTRACT

Novel small molecule proteolysis-targeting chimeras (PROTACs) are provided, along with methods for their use as Bruton's tyrosine kinase (BTK) degraders. The small molecule PROTACs described herein are useful in treating and/or preventing BTK-related diseases, such as cancer, neurodegenerative disorders, inflammatory diseases, and metabolic disorders. Also provided are methods for inducing BTK degradation in a cell using the compounds and compositions described herein.

### CROSS-REFERENCE TO PRIORITY APPLICATION

This application claims priority to U.S. Provisional Application No. 63/502,209, filed May 15, 2023, which is incorporated herein by reference in its entirety.

### STATEMENT REGARDING FEDERALLY FUNDED RESEARCH

This invention was made with government support under Grant Nos. R01-GM115622 and R01-CA250503, awarded by the National Institutes of Health. The government has certain rights in the invention.

### BACKGROUND

Bruton's Tyrosine Kinase (BTK) is a Tec-family tyrosine kinase present in all blood cells except for T cells and natural killer cells. Overexpression of BTK is associated with various B cell malignancies, including mantle cell lymphoma (MCL) and chronic lymphocytic leukemia (CLL). BTK is a clinically proven target to attenuate B cell receptor (BCR) signaling and induce cell death in these cancer cells. Several BTK kinase inhibitors have been approved to treat B cell malignancies. Not only serving as a kinase, BTK can also enhance antigen receptor-induced calcium influx in a kinase-independent manner. Therefore, developing BTK degraders to abolish both kinase-dependent and -independent functions attracts significant interests in academia and industry.

### SUMMARY

Described herein are compounds of the following formula:

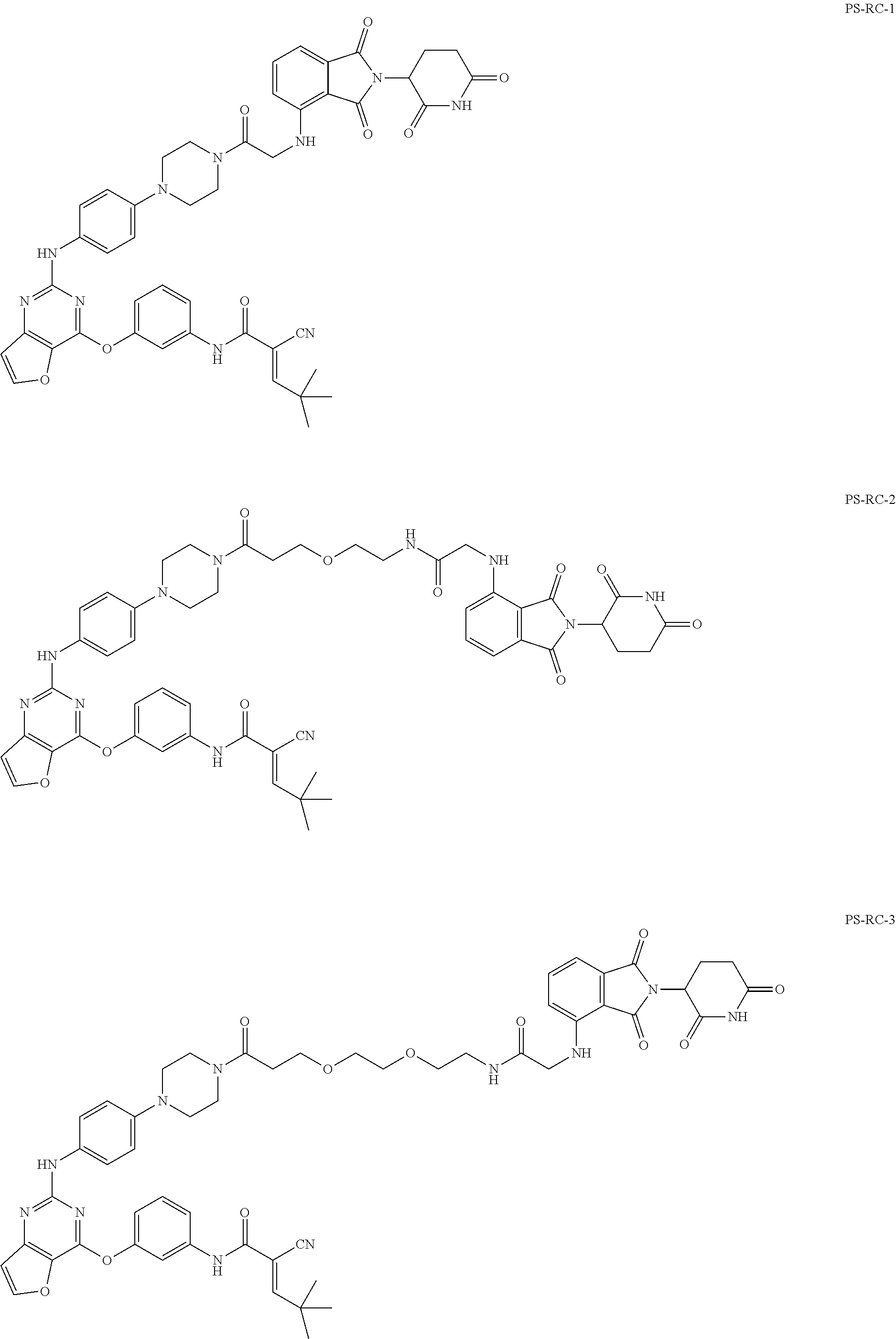 [SMILES: CC(C)(C)/C=C(\C#N)C(=O)Nc1cccc(Oc2nc(Nc3ccc(N4CCN(C(=O)CNc5cccc6c5C(=O)N(C5CCC(=O)NC5=O)C6=O)CC4)cc3)nc3ccoc23)c1 ; CC(C)(C)/C=C(\C#N)C(=O)Nc1cccc(Oc2nc(Nc3ccc(N4CCN(C(=O)CCOCCNC(=O)CNc5cccc6c5C(=O)N(C5CCC(=O)NC5=O)C6=O)CC4)cc3)nc3ccoc23)c1 ; CC(C)(C)/C=C(\C#N)C(=O)Nc1cccc(Oc2nc(Nc3ccc(N4CCN(C(=O)CCOCCOCCNC(=O)CNc5cccc6c5C(=O)N(C5CCC(=O)NC5=O)C6=O)CC4)cc3)nc3ccoc23)c1]

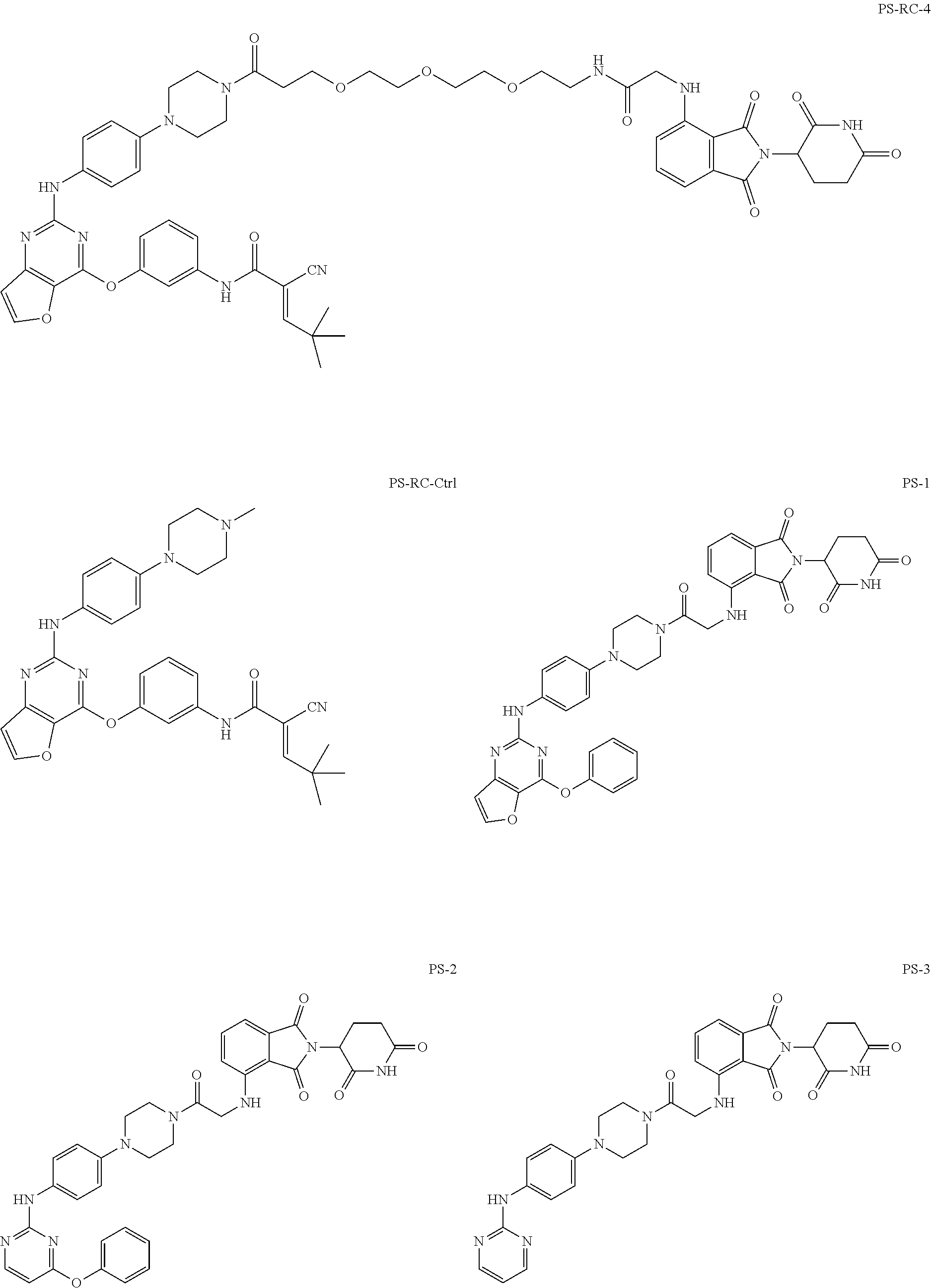 [SMILES: CC(C)(C)/C=C(\C#N)C(=O)Nc1cccc(Oc2nc(Nc3ccc(N4CCN(C(=O)CCOCCOCCOCCNC(=O)CNc5cccc6c5C(=O)N(C5CCC(=O)NC5=O)C6=O)CC4)cc3)nc3ccoc23)c1 ; CN1CCN(c2ccc(Nc3nc(Oc4cccc(NC(=O)/C(C#N)=C/C(C)(C)C)c4)c4occc4n3)cc2)CC1 ; O=C1CCC(N2C(=O)c3cccc(NCC(=O)N4CCN(c5ccc(Nc6nc(Oc7ccccc7)c7occc7n6)cc5)CC4)c3C2=O)C(=O)N1 ; O=C1CCC(N2C(=O)c3cccc(NCC(=O)N4CCN(c5ccc(Nc6nccc(Oc7ccccc7)n6)cc5)CC4)c3C2=O)C(=O)N1 ; O=C1CCC(N2C(=O)c3cccc(NCC(=O)N4CCN(c5ccc(Nc6ncccn6)cc5)CC4)c3C2=O)C(=O)N1]

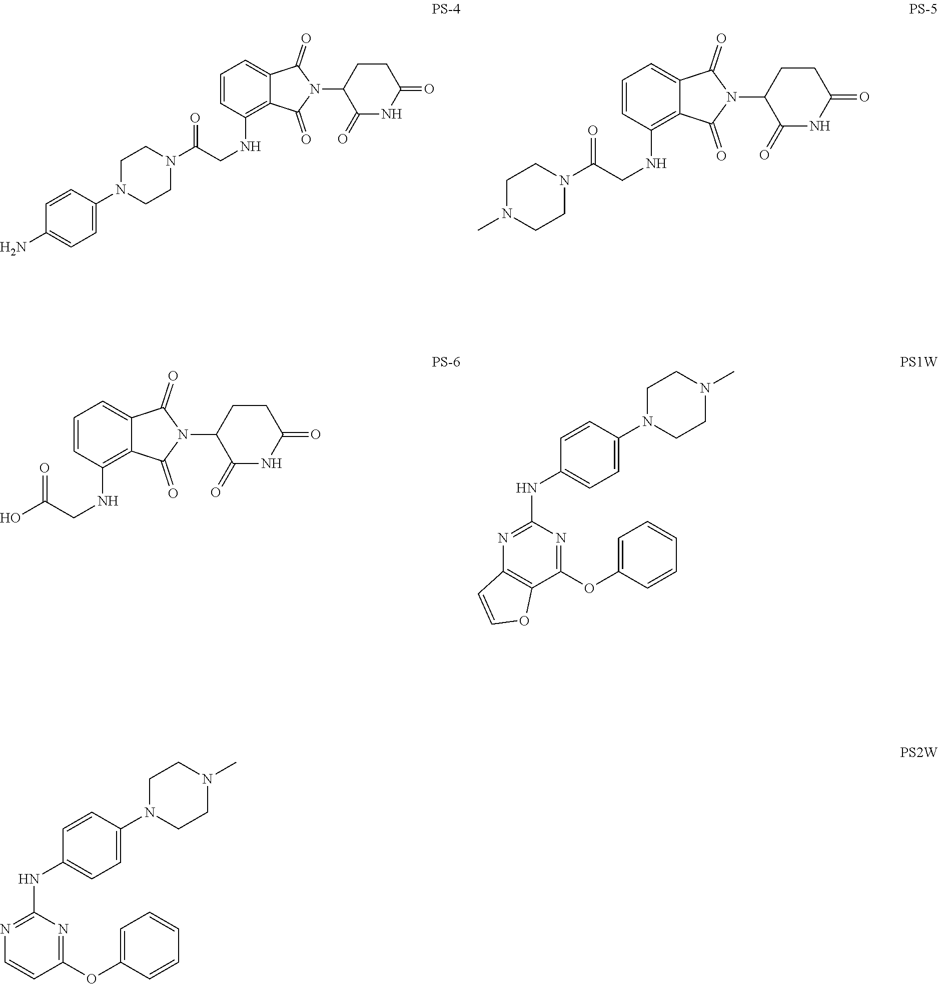 [SMILES: Nc1ccc(N2CCN(C(=O)CNc3cccc4c3C(=O)N(C3CCC(=O)NC3=O)C4=O)CC2)cc1 ; CN1CCN(C(=O)CNc2cccc3c2C(=O)N(C2CCC(=O)NC2=O)C3=O)CC1 ; O=C(O)CNc1cccc2c1C(=O)N(C1CCC(=O)NC1=O)C2=O ; CN1CCN(c2ccc(Nc3nc(Oc4ccccc4)c4occc4n3)cc2)CC1 ; CN1CCN(c2ccc(Nc3nccc(Oc4ccccc4)n3)cc2)CC1]

or a pharmaceutically acceptable salt or prodrug thereof. Also described herein are compositions comprising a compound as described above and a pharmaceutically acceptable carrier, along with kits comprising the compounds and compositions described herein.

Also described herein are methods of treating or preventing a BTK-related disease in a subject, comprising administering to the subject an effective amount of a compound or composition as described herein. Optionally, the BTK-related disease is cancer (e.g., bladder cancer, blood cancer, a bone marrow cancer, brain cancer, breast cancer, bronchus cancer, colorectal cancer, cervical cancer, chondrosarcoma, endometrial cancer, gastrointestinal cancer, gastric cancer, genitourinary cancer, head and neck cancer, hepatic cancer, hepatocellular carcinoma, leukemia, liver cancer, lung cancer, lymphoma, melanoma of the skin, ovarian cancer, pancreatic cancer, prostate cancer, renal cancer, skin cancer, testicular cancer, thyroid cancer, or uterine cancer). Optionally, the BTK-related disease is a neurodegenerative disorder or an inflammatory disease. The methods described herein can further include administering a second compound, biomolecule, or composition. Optionally, the second compound, biomolecule, or composition comprises a chemotherapeutic agent.

Further described herein are methods of inducing BTK degradation in a cell, comprising contacting a cell with an effective amount of a compound as described herein. The contacting can be performed in vitro or in vivo.

The details of one or more embodiments are set forth in the drawings and the description below. Other features, objects, and advantages will be apparent from the description and drawings, and from the claims.

### DESCRIPTION OF DRAWINGS

FIG. 1 contains the chemical structures of compounds described herein.

FIG. 2 shows that poseltinib-based reversible covalent BTK PROTACs cannot induce BTK degradation in cells Panels a-e: Mino cells were treated with indicated compounds at 0, 1, 6, 8, 40, 200, and 1000 nM for 24 h, followed by Western blotting for BTK PS-RC-1, PS-RC-2, PS-RC-3, and PS-RC-4 are poseltinib-based reversible covalent BTK PROTACs. DD-03-171 is a known BTK degrader and used as a positive control. Panel f: HEK-293T cells stably expressing a BTK-nLuc fusion protein were treated with indicated compounds (same as in a-e) for 24 h. The BTK degradation was determined by evaluating luminescence signals of NanoLuc.

FIG. 3 shows the toxicities of poseltinib-based BTK PROTACs in cells and their binding affinities to BTK. Panels a-b. MOLM14 and Mino cells were treated with serially diluted poseltinib and PS-RC-1 to PS-RC-4 for 72 h, followed by Alarma Blue assay to quantify the cell viabilities. Panel c. Three MCL cell lines, including Mino, Jeko-1, and Rec-R cells, were treated with serially diluted PS-RC-1 for 72 h, followed by Alarma Blue assay to quantify the cell viabilities. Panel d. TR-FRET based binding kinetics assay between poseltinib and BTK. Serial dilutions of poseltinib mixed with 2 nM of His-BTK, 0.3 nM Tb-anti-His, and 150 nM of BTK-BODIPY tracer. Panel e. BTK binding affinity assays for poseltinib-based PROTACs (PS-RC-1 to PS-RC-4), following the same protocol as described for Panel d. After 2 h incubation, TR-FRET signals were measured. The IC₅₀ values were listed in Table 1. Panel f. PS-RC-1 serves as a molecular glue to inhibit growth in Mino cells. Mino cells were pre-treated with a large excess of PS-RC-Ctrl (2 μM or 10 μM), followed by PS-RC-1 incubation for 72 h. The cell viabilities were quantified using an Alarma Blue assay. For cell viability assays, data represent mean±SD (n=3) and the IC₅₀ values are defined as compound concentrations that reduce cell viabilities by 50%. For BTK binding assays, data represent mean±SD (n=3) and the IC₅₀ values are defined as compound concentrations that reduce tracer binding by 50%.

FIG. 4 shows that PS-1 and PS-2 potently degrades IKZF1 and IKZF3 and showed significant toxicity in Mino cells. Panel a. Mino cells were treated with PS-RC-1 or CC-885 at 0, 1.6, 8, 40, 200, and 1000 nM for 24 h, followed by Western blotting for GSPT1. In contrast to the potent GSPT1 degrader CC-885, PS-RC-1 does not degrade GSPT1. Panel b. Mino cells were treated with indicated compounds at 0, 1.6, 8, 40, 200, and 1000 nM for 24 h, followed by Western blotting for IKZF1 and IKZF3. Panel c. HEK-293T-IKZF1/IKZF3-nLuc cells were treated with indicated compounds for 24 h and the degradation of IKZF1/IKZF3 were determined by measuring Nanoluc luminescence signals. Data represent mean±SD (n=3) and the DC₅₀ values are defined as compound concentrations that reduce nLuc signals by 50%. Panel d-e. Mino and MM.1S cells were treated with serially diluted indicated compounds for 72 and 120 h, respectively, followed by Alarma Blue assay to quantify the cell viabilities. For cell viability assays, data represent mean±SD (n=3 and the IC₅₀ values are defined as compound concentrations that reduce cell viabilities by 50%. Panel f. TMT-based quantitative proteome analysis of protein expression changes from PS-2 (200 nM) treatment for 4 h in Mino cells. Volcano plot shows protein abundance (log 2) as a function of significance level (log 10). The vertical dotted line denotes 25% reduction of protein levels, whereas the horizontal dotted lines marks p=0.01 and p=0.05 statistical significance thresholds. Downregulated proteins with statistical significance are found in the upper left quadrant of the plots. Dataset represents an average of n=3 replicates. A total of 2,543 proteins were identified, and only the ones with at least one uniquely identified peptide are plotted. Panel g. Degradation kinetics of IKZF1 and IKZF3. HEK-293T-IKZF1/IKZF3-nLuc cells were treated with indicated compounds, followed by immediate measurements of Nanoluc luminescence signals every 5 min for 24 h. The degradation kinetics were fitted into a mono-exponential decay model.

FIG. 5 shows that PS-2 induces BTK degradation in cells. Mino cells were treated with indicated compounds at 0, 1.6, 8, 40, 200, and 1000 nM for 24 h, Ramos and A20 cells were incubated with PS-1, PS-2, PS-3, and DD-03-171 at 40, 200, and 1000 nM for 24 h, followed by Western blotting for BTK. CC-220 is a IKZF1/3 degrader developed by Celgene and used for comparison. In contrast to PS-1, PS-3 and CC-220, PS-2 potently induces BTK degradation in Mino cells. In Ramos and A20 cell lines, PS-1 and PS-3 elicit very weak or no BTK degradation, while PS-2 induces potent BTK degradation that is comparable to DD-03-171.

FIG. 6 shows PS-2 induced BTK degradation in Ramos cells. Ramos cells were treated with the indicated compounds at the indicated concentrations for 24 hours. Nurix and DD-03-171 were used as the controls.

FIG. 7 shows the results from a BTK ligand competition assay. Ramos cells were incubated overnight, and then the indicated compounds were added and incubated for 24 hours, followed by Western blotting for BTK degradation.

FIG. 8 shows the results from a CRBN ligand competition assay. Ramos cells were incubated overnight, and then the indicated compounds were added and incubated for 24 hours, followed by Western blotting for BTK degradation.

FIG. 9 shows the results from binding affinity assays for BTK.

FIG. 10 shows pharmacodynamic data results. Mice were intraperitoneally administered PS-2 at a dose of 30 mg/kg once a day for three days. Four mice were tested per drug.

FIG. 11 shows mouse S9 metabolic stability assay data for compound PS-2.

### DETAILED DESCRIPTION

Described herein are novel small molecule proteolysis-targeting chimeras (PROTACs) and methods for their use as Bruton's tyrosine kinase (BTK) degraders. A proteolysis targeting chimera (PROTAC) is a heterobifunctional molecule that can bind both a targeted protein and an E3 ubiquitin ligase to facilitate the formation of a ternary complex, leading to ubiquitination and ultimate degradation of the target protein. Using the PROTAC approach, described herein are orally available BTK degraders. The reversible covalent BTK PROTACs described herein are highly potent and selective. The compounds described herein are capable of selectivity inducing degradation of IKZF1 and IKZF3. In addition, lower molecular weight compounds are described herein along with their uses as potent triple degraders of BTK and lymphoid transcription factors Ikaros (IKZF1) and Aiolos (IKZF3). The small molecule PROTACs described herein are useful in treating and/or preventing cancer, neurodegenerative disorders, inflammatory diseases, metabolic disorders, and other BTK-related diseases.

### I. Compounds

In some cases, the PROTACs described herein are compounds as shown below:

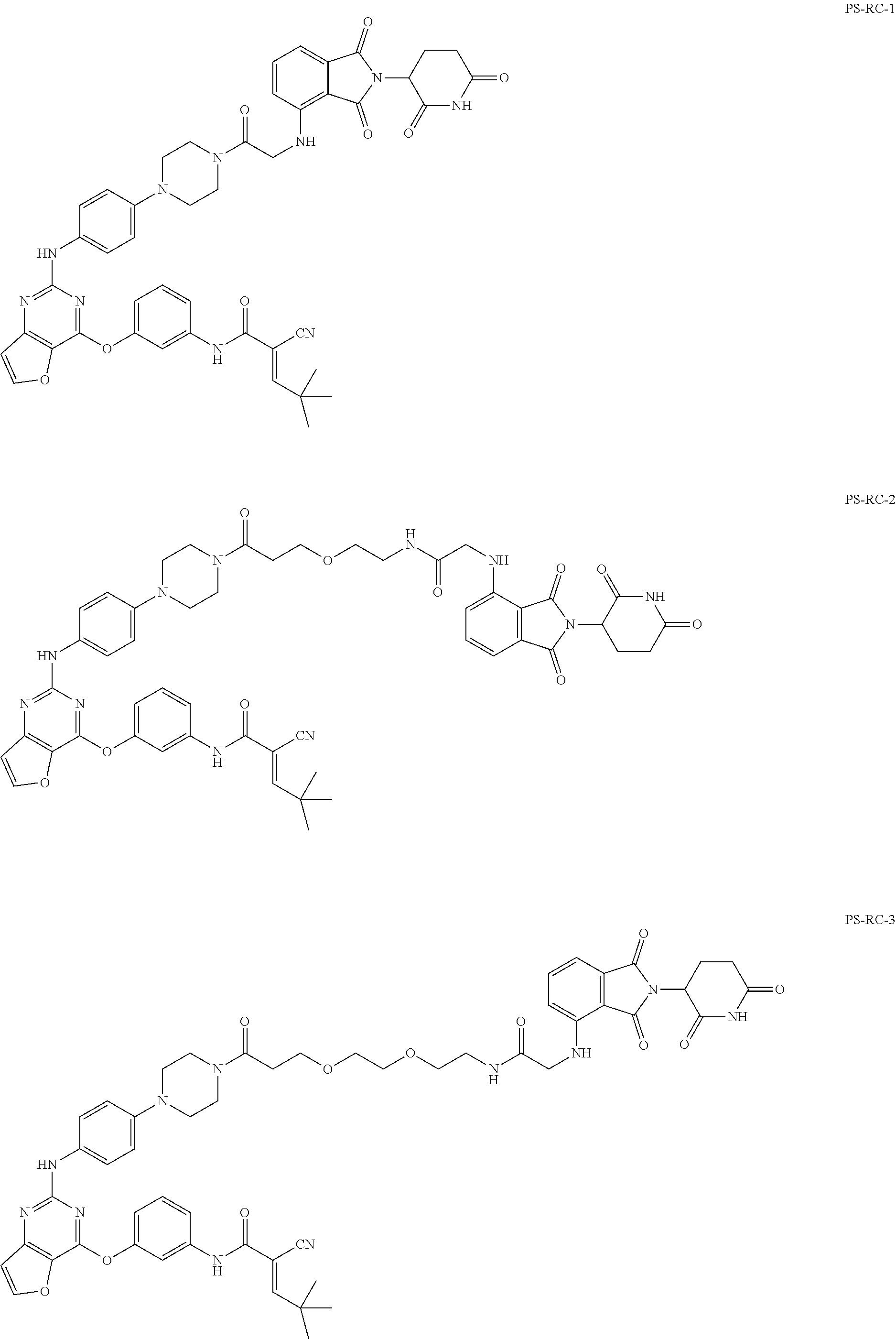 [SMILES: CC(C)(C)/C=C(\C#N)C(=O)Nc1cccc(Oc2nc(Nc3ccc(N4CCN(C(=O)CNc5cccc6c5C(=O)N(C5CCC(=O)NC5=O)C6=O)CC4)cc3)nc3ccoc23)c1 ; CC(C)(C)/C=C(\C#N)C(=O)Nc1cccc(Oc2nc(Nc3ccc(N4CCN(C(=O)CCOCCNC(=O)CNc5cccc6c5C(=O)N(C5CCC(=O)NC5=O)C6=O)CC4)cc3)nc3ccoc23)c1 ; CC(C)(C)/C=C(\C#N)C(=O)Nc1cccc(Oc2nc(Nc3ccc(N4CCN(C(=O)CCOCCOCCNC(=O)CNc5cccc6c5C(=O)N(C5CCC(=O)NC5=O)C6=O)CC4)cc3)nc3ccoc23)c1]

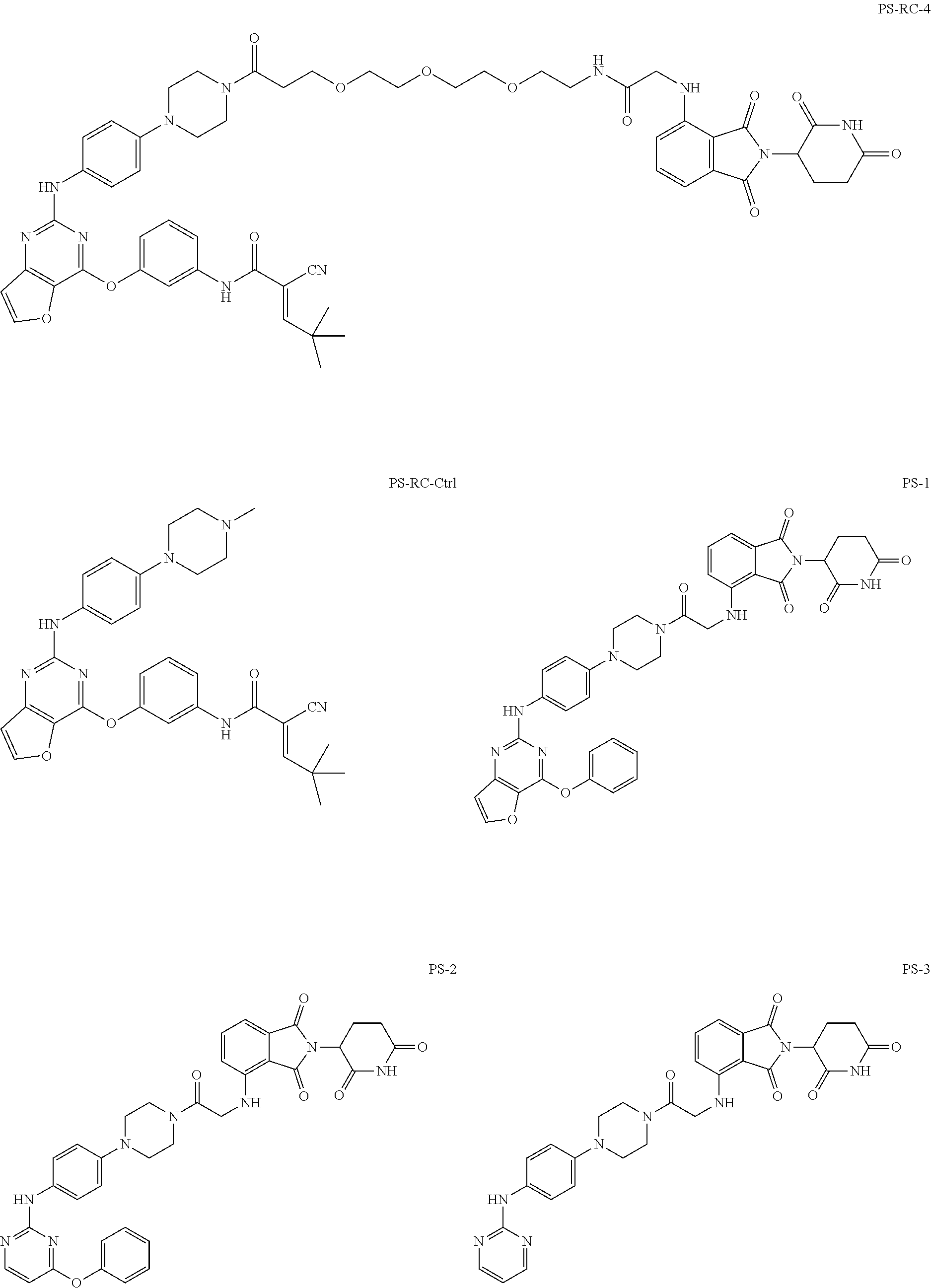 [SMILES: CC(C)(C)/C=C(\C#N)C(=O)Nc1cccc(Oc2nc(Nc3ccc(N4CCN(C(=O)CCOCCOCCOCCNC(=O)CNc5cccc6c5C(=O)N(C5CCC(=O)NC5=O)C6=O)CC4)cc3)nc3ccoc23)c1 ; CN1CCN(c2ccc(Nc3nc(Oc4cccc(NC(=O)/C(C#N)=C/C(C)(C)C)c4)c4occc4n3)cc2)CC1 ; O=C1CCC(N2C(=O)c3cccc(NCC(=O)N4CCN(c5ccc(Nc6nc(Oc7ccccc7)c7occc7n6)cc5)CC4)c3C2=O)C(=O)N1 ; O=C1CCC(N2C(=O)c3cccc(NCC(=O)N4CCN(c5ccc(Nc6nccc(Oc7ccccc7)n6)cc5)CC4)c3C2=O)C(=O)N1 ; O=C1CCC(N2C(=O)c3cccc(NCC(=O)N4CCN(c5ccc(Nc6ncccn6)cc5)CC4)c3C2=O)C(=O)N1]

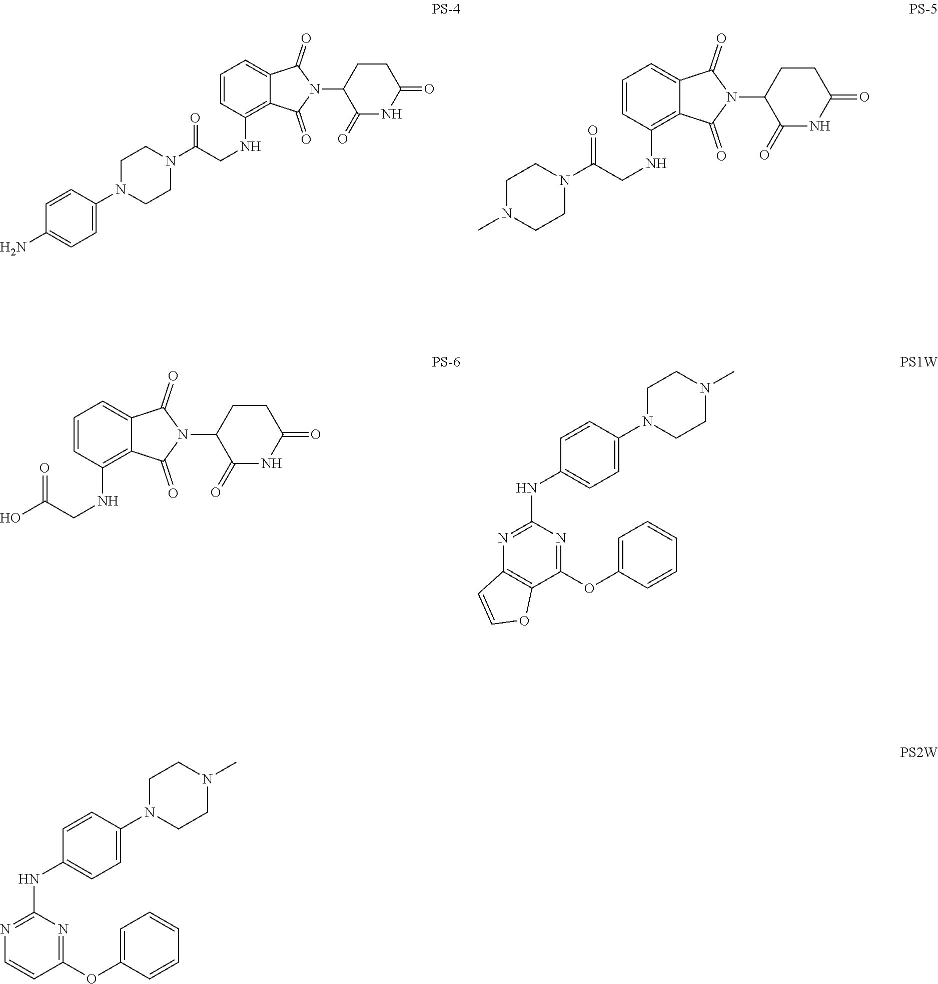 [SMILES: Nc1ccc(N2CCN(C(=O)CNc3cccc4c3C(=O)N(C3CCC(=O)NC3=O)C4=O)CC2)cc1 ; CN1CCN(C(=O)CNc2cccc3c2C(=O)N(C2CCC(=O)NC2=O)C3=O)CC1 ; O=C(O)CNc1cccc2c1C(=O)N(C1CCC(=O)NC1=O)C2=O ; CN1CCN(c2ccc(Nc3nc(Oc4ccccc4)c4occc4n3)cc2)CC1 ; CN1CCN(c2ccc(Nc3nccc(Oc4ccccc4)n3)cc2)CC1]

An additional compound described herein, optionally for use in the methods described herein includes the following:

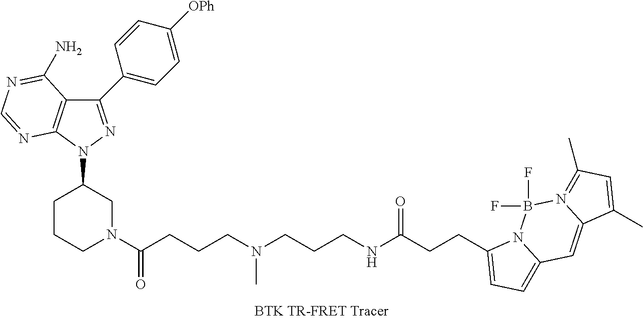

The compounds described herein also include isotopic substitutions (e.g., a deuterium or tritium variant) of the compounds. In particular, one or more hydrogen atoms can be substituted by a hydrogen isotope (e.g., a deuterium or a tritium). For example, a methoxy group (—OCH₃) can be substituted with one or more isotopic groups to form, for example, —OCDH₂, —OCD₂H, or —OCD₃.

### II. Methods of Making the Compounds

The compounds described herein can be prepared in a variety of ways. The compounds can be synthesized using various synthetic methods. At least some of these methods are known in the art of synthetic organic chemistry. The compounds described herein can be prepared from readily available starting materials. Optimum reaction conditions can vary with the particular reactants or solvent used, but such conditions can be determined by one skilled in the art by routine optimization procedures.

Variations on the compounds described herein include the addition, subtraction, or movement of the various constituents as described for each compound. Similarly, when one or more chiral centers are present in a molecule, all possible chiral variants are included.

Additionally, compound synthesis can involve the protection and deprotection of various chemical groups. The use of protection and deprotection, and the selection of appropriate protecting groups can be determined by one skilled in the art. The chemistry of protecting groups can be found, for example, in Wuts, Greene's Protective Groups in Organic Synthesis, 5th. Ed., Wiley & Sons, 2014, which is incorporated herein by reference in its entirety.

Reactions to produce the compounds described herein can be carried out in solvents, which can be selected by one of skill in the art of organic synthesis. Solvents can be substantially nonreactive with the starting materials (reactants), the intermediates, or products under the conditions at which the reactions are carried out, i.e., temperature and pressure.

Reactions can be carried out in one solvent or a mixture of more than one solvent. Product or intermediate formation can be monitored according to any suitable method known in the art. For example, product formation can be monitored by spectroscopic means, such as nuclear magnetic resonance spectroscopy (e.g., ¹H or ¹³C) infrared spectroscopy, spectrophotometry (e.g., UV-visible), or mass spectrometry, or by chromatography such as high-performance liquid chromatography (HPLC) or thin layer chromatography.

Exemplary methods for synthesizing compounds as described herein are provided in Example 1 below.

### III. Pharmaceutical Formulations

The compounds described herein or derivatives thereof can be provided in a pharmaceutical composition. Depending on the intended mode of administration, the pharmaceutical composition can be in the form of solid, semi-solid or liquid dosage forms, such as, for example, tablets, suppositories, pills, capsules, powders, liquids, or suspensions, preferably in unit dosage form suitable for single administration of a precise dosage. The compositions will include a therapeutically effective amount of the compound described herein or derivatives thereof in combination with a pharmaceutically acceptable carrier and, in addition, may include other medicinal agents, pharmaceutical agents, carriers, or diluents. By pharmaceutically acceptable is meant a material that is not biologically or otherwise undesirable, which can be administered to an individual along with the selected compound without causing unacceptable biological effects or interacting in a deleterious manner with the other components of the pharmaceutical composition in which it is contained.

As used herein, the term carrier encompasses any excipient, diluent, filler, salt, buffer, stabilizer, solubilizer, lipid, stabilizer, or other material well known in the art for use in pharmaceutical formulations. The choice of a carrier for use in a composition will depend upon the intended route of administration for the composition. The preparation of pharmaceutically acceptable carriers and formulations containing these materials is described in, e.g., Remington: The Science and Practice of Pharmacy, 22d Edition, Loyd et al. eds., Pharmaceutical Press and Philadelphia College of Pharmacy at University of the Sciences (2012). Examples of physiologically acceptable carriers include buffers, such as phosphate buffers, citrate buffer, and buffers with other organic acids; antioxidants including ascorbic acid; low molecular weight (less than about 10 residues) polypeptides; proteins, such as serum albumin, gelatin, or immunoglobulins; hydrophilic polymers, such as polyvinylpyrrolidone; amino acids such as glycine, glutamine, asparagine, arginine or lysine; monosaccharides, disaccharides, and other carbohydrates, including glucose, mannose, or dextrins; chelating agents, such as EDTA; sugar alcohols, such as mannitol or sorbitol; salt-forming counterions, such as sodium; and/or nonionic surfactants, such as TWEEN® (ICI, Inc.; Bridgewater, New Jersey), polyethylene glycol (PEG), and PLURONICS™ (BASF; Florham Park, NJ).

Compositions containing the compound described herein or derivatives thereof suitable for parenteral injection may comprise physiologically acceptable sterile aqueous or nonaqueous solutions, dispersions, suspensions or emulsions, and sterile powders for reconstitution into sterile injectable solutions or dispersions. Examples of suitable aqueous and nonaqueous carriers, diluents, solvents or vehicles include water, ethanol, polyols (propyleneglycol, polyethyleneglycol, glycerol, and the like), suitable mixtures thereof, vegetable oils (such as olive oil) and injectable organic esters such as ethyl oleate. Proper fluidity can be maintained, for example, by the use of a coating such as lecithin, by the maintenance of the required particle size in the case of dispersions and by the use of surfactants.

These compositions may also contain adjuvants, such as preserving, wetting, emulsifying, and dispensing agents. Prevention of the action of microorganisms can be promoted by various antibacterial and antifungal agents, for example, parabens, chlorobutanol, phenol, sorbic acid, and the like. Isotonic agents, for example, sugars, sodium chloride, and the like may also be included. Prolonged absorption of the injectable pharmaceutical form can be brought about by the use of agents delaying absorption, for example, aluminum monostearate and gelatin.

Solid dosage forms for oral administration of the compounds described herein or derivatives thereof include capsules, tablets, pills, powders, and granules. In such solid dosage forms, the compounds described herein or derivatives thereof is admixed with at least one inert customary excipient (or carrier), such as sodium citrate or dicalcium phosphate, or (a) fillers or extenders, as for example, starches, lactose, sucrose, glucose, mannitol, and silicic acid, (b) binders, as for example, carboxymethylcellulose, alignates, gelatin, polyvinylpyrrolidone, sucrose, and acacia, (c) humectants, as for example, glycerol, (d) disintegrating agents, as for example, agar-agar, calcium carbonate, potato or tapioca starch, alginic acid, certain complex silicates, and sodium carbonate, (e) solution retarders, as for example, paraffin, (f) absorption accelerators, as for example, quaternary ammonium compounds, (g) wetting agents, as for example, cetyl alcohol, and glycerol monostearate, (h) adsorbents, as for example, kaolin and bentonite, and (i) lubricants, as for example, talc, calcium stearate, magnesium stearate, solid polyethylene glycols, sodium lauryl sulfate, or mixtures thereof. In the case of capsules, tablets, and pills, the dosage forms may also comprise buffering agents.

Solid compositions of a similar type may also be employed as fillers in soft and hard-filled gelatin capsules using such excipients as lactose or milk sugar as well as high molecular weight polyethyleneglycols, and the like.

Solid dosage forms such as tablets, dragees, capsules, pills, and granules can be prepared with coatings and shells, such as enteric coatings and others known in the art. They may contain opacifying agents and can also be of such composition that they release the active compound or compounds in a certain part of the intestinal tract in a delayed manner. Examples of embedding compositions that can be used are polymeric substances and waxes. The active compounds can also be in micro-encapsulated form, if appropriate, with one or more of the above-mentioned excipients.

Liquid dosage forms for oral administration of the compounds described herein or derivatives thereof include pharmaceutically acceptable emulsions, solutions, suspensions, syrups, and elixirs. In addition to the active compounds, the liquid dosage forms may contain inert diluents commonly used in the art, such as water or other solvents, solubilizing agents, and emulsifiers, as for example, ethyl alcohol, isopropyl alcohol, ethyl carbonate, ethyl acetate, benzyl alcohol, benzyl benzoate, propyleneglycol, 1,3-butyleneglycol, dimethylformamide, oils, in particular, cottonseed oil, groundnut oil, corn germ oil, olive oil, castor oil, sesame oil, glycerol, tetrahydrofurfuryl alcohol, polyethyleneglycols, and fatty acid esters of sorbitan, or mixtures of these substances, and the like.

Besides such inert diluents, the composition can also include additional agents, such as wetting, emulsifying, suspending, sweetening, flavoring, or perfuming agents.

Suspensions, in addition to the active compounds, may contain additional agents, as for example, ethoxylated isostearyl alcohols, polyoxyethylene sorbitol and sorbitan esters, microcrystalline cellulose, aluminum metahydroxide, bentonite, agar-agar and tragacanth, or mixtures of these substances, and the like.

Compositions of the compounds described herein or derivatives thereof for rectal administrations are optionally suppositories, which can be prepared by mixing the compounds with suitable non-irritating excipients or carriers, such as cocoa butter, polyethyleneglycol or a suppository wax, which are solid at ordinary temperatures but liquid at body temperature and, therefore, melt in the rectum or vaginal cavity and release the active component.

Dosage forms for topical administration of the compounds described herein or derivatives thereof include ointments, powders, sprays, inhalants, and skin patches. The compounds described herein or derivatives thereof are admixed under sterile conditions with a physiologically acceptable carrier and any preservatives, buffers, or propellants as may be required. Ophthalmic formulations, ointments, powders, and solutions are also contemplated as being within the scope of the compositions.

Optionally, the compounds described herein can be contained in a drug depot. A drug depot comprises a physical structure to facilitate implantation and retention in a desired site (e.g., a synovial joint, a disc space, a spinal canal, abdominal area, a tissue of the patient, etc.). The drug depot can provide an optimal concentration gradient of the compound at a distance of up to about 0.1 cm to about 5 cm from the implant site. A depot, as used herein, includes but is not limited to capsules, microspheres, microparticles, microcapsules, microfibers particles, nanospheres, nanoparticles, coating, matrices, wafers, pills, pellets, emulsions, liposomes, micelles, gels, antibody-compound conjugates, protein-compound conjugates, or other pharmaceutical delivery compositions. Suitable materials for the depot include pharmaceutically acceptable biodegradable materials that are preferably FDA approved or GRAS materials. These materials can be polymeric or non-polymeric, as well as synthetic or naturally occurring, or a combination thereof. The depot can optionally include a drug pump.

The compositions can include one or more of the compounds described herein and a pharmaceutically acceptable carrier. As used herein, the term pharmaceutically acceptable salt refers to those salts of the compound described herein or derivatives thereof that are, within the scope of sound medical judgment, suitable for use in contact with the tissues of subjects without undue toxicity, irritation, allergic response, and the like, commensurate with a reasonable benefit/risk ratio, and effective for their intended use, as well as the zwitterionic forms, where possible, of the compounds described herein. The term salts refers to the relatively non-toxic, inorganic and organic acid addition salts of the compounds described herein. These salts can be prepared in situ during the isolation and purification of the compounds or by separately reacting the purified compound in its free base form with a suitable organic or inorganic acid and isolating the salt thus formed. Representative salts include the hydrobromide, hydrochloride, sulfate, bisulfate, nitrate, acetate, oxalate, valerate, oleate, palmitate, stearate, laurate, borate, benzoate, lactate, phosphate, tosylate, citrate, maleate, fumarate, succinate, tartrate, naphthylate mesylate, glucoheptonate, lactobionate, methane sulphonate, and laurylsulphonate salts, and the like. These may include cations based on the alkali and alkaline earth metals, such as sodium, lithium, potassium, calcium, magnesium, and the like, as well as non-toxic ammonium, quaternary ammonium, and amine cations including, but not limited to ammonium, tetramethylammonium, tetraethylammonium, methylamine, dimethylamine, trimethylamine, triethylamine, ethylamine, and the like. (See S. M. Barge et al., J. Pharm. Sci. (1977) 66, 1, which is incorporated herein by reference in its entirety, at least, for compositions taught therein.)

Administration of the compounds and compositions described herein or pharmaceutically acceptable salts thereof can be carried out using therapeutically effective amounts of the compounds and compositions described herein or pharmaceutically acceptable salts thereof as described herein for periods of time effective to treat a disorder. The effective amount of the compounds and compositions described herein or pharmaceutically acceptable salts thereof as described herein may be determined by one of ordinary skill in the art and includes exemplary dosage amounts for a mammal of from about 0.0001 to about 200 mg/kg of body weight of active compound per day, which may be administered in a single dose or in the form of individual divided doses, such as from 1 to 4 times per day. Alternatively, the dosage amount can be from about 0.01 to about 150 mg/kg of body weight of active compound per day, about 0.1 to 100 mg/kg of body weight of active compound per day, about 0.5 to about 75 mg/kg of body weight of active compound per day, about 0.5 to about 50 mg/kg of body weight of active compound per day, about 0.01 to about 50 mg/kg of body weight of active compound per day, about 0.05 to about 25 mg/kg of body weight of active compound per day, about 0.1 to about 25 mg/kg of body weight of active compound per day, about 0.5 to about 25 mg/kg of body weight of active compound per day, about 1 to about 20 mg/kg of body weight of active compound per day, about 1 to about 10 mg/kg of body weight of active compound per day, about 20 mg/kg of body weight of active compound per day, about 10 mg/kg of body weight of active compound per day, about 5 mg/kg of body weight of active compound per day, about 2.5 mg/kg of body weight of active compound per day, about 1.0 mg/kg of body weight of active compound per day, or about 0.5 mg/kg of body weight of active compound per day, or any range derivable therein. Optionally, the dosage amounts are from about 0.01 mg/kg to about 10 mg/kg of body weight of active compound per day. Optionally, the dosage amount is from about 0.01 mg/kg to about 5 mg/kg. Optionally, the dosage amount is from about 0.01 mg/kg to about 2.5 mg/kg.

Those of skill in the art will understand that the specific dose level and frequency of dosage for any particular subject may be varied and will depend upon a variety of factors, including the activity of the specific compound employed, the metabolic stability and length of action of that compound, the species, age, body weight, general health, sex and diet of the subject, the mode and time of administration, rate of excretion, drug combination, and severity of the particular condition.

The precise dose to be employed in the formulation will also depend on the route of administration, and the seriousness of the disease or disorder, and should be decided according to the judgment of the practitioner and each subject's circumstances. Effective doses can be extrapolated from dose-response curves derived from in vitro or animal model test systems. Further, depending on the route of administration, one of skill in the art would know how to determine doses that result in a plasma concentration for a desired level of response in the cells, tissues and/or organs of a subject.

### IV. Methods of Use

Provided herein are methods to treat, prevent, or ameliorate a BTK-related disease in a subject. The methods include administering to a subject an effective amount of one or more of the compounds or compositions described herein, or a pharmaceutically acceptable salt or prodrug thereof. Effective amount, when used to describe an amount of compound in a method, refers to the amount of a compound that achieves the desired pharmacological effect or other biological effect. The effective amount can be, for example, the concentrations of compounds at which BTK is degraded in vitro, as provided herein. Also contemplated is a method that includes administering to the subject an amount of one or more compounds described herein such that an in vivo concentration at a target cell in the subject corresponding to the concentration administered in vitro is achieved.

The compounds and compositions described herein or pharmaceutically acceptable salts thereof are useful for treating BTK-related diseases in humans, including, without limitation, pediatric and geriatric populations, and in animals, e.g., veterinary applications.

In some embodiments, the BTK-related disease is cancer. Optionally, the cancer is a poor prognosis cancer. The term poor prognosis, as used herein, refers to a prospect of recovery from a disease, infection, or medical condition that is associated with a diminished likelihood of a positive outcome. In relation to a disease such as cancer, a poor prognosis may be associated with a reduced patient survival rate, reduced patient survival time, higher likelihood of metastatic progression of said cancer cells, and/or higher likelihood of chemoresistance of said cancer cells. Optionally, a poor prognosis cancer can be a cancer associated with a patient survival rate of 50% or less. Optionally, a poor prognosis cancer can be a cancer associated with a patient survival time of five years or less after diagnosis. In some embodiments, the cancer is an invasive cancer.

Optionally, the cancer is a cancer that has an increased expression of BTK as compared to non-cancerous cells of the same cell type. Optionally, the cancer is bladder cancer, brain cancer, breast cancer (e.g., triple negative breast cancer), bronchus cancer, colorectal cancer (e.g., colon cancer, rectal cancer), cervical cancer, chondrosarcoma, endometrial cancer, gastrointestinal cancer, gastric cancer, genitourinary cancer, glioblastoma, head and neck cancer, hepatic cancer, hepatocellular carcinoma, leukemia, liver cancer, lung cancer, lymphoma, melanoma of the skin, ovarian cancer, pancreatic cancer, prostate cancer, renal cancer, skin cancer, testicular cancer, thyroid cancer, or uterine cancer. Optionally, the cancer is a cancer that affects one or more of the following sites: oral cavity and pharynx (e.g., tongue, mouth, pharynx, or other oral cavity); digestive system (e.g., esophagus, stomach, small intestine, colon, rectum, anus, anal canal, anorectum, liver and intrahepatic bile duct, gallbladder and other biliary, pancreas, or other digestive organs); respiratory system (e.g., larynx, lung and bronchus, or other respiratory organs); bones and joints; soft tissue (e.g., heart); skin (e.g., melanoma of the skin or other nonepithelial skin); breast; genital system (e.g., uterine cervix, uterine corpus, ovary, vulva, vagina and other female genital areas, prostate, testis, penis and other male genital areas); urinary system (e.g., urinary bladder, kidney and renal pelvis, and ureter and other urinary organs); eye and orbit; brain and other nervous system; endocrine system (e.g., thyroid and other endocrine); lymphoma (e.g., Hodgkin lymphoma and non-Hodgkin lymphoma); myeloma; or leukemia (e.g., acute lymphocytic leukemia, chronic lymphocytic leukemia, acute myeloid leukemia, chronic myeloid leukemia, or other leukemia). Optionally, the cancer is a drug resistant cancer, such as an ibrutinib-resistant cancer.

In some embodiments, the BTK-related disease is a metabolic disorder (e.g., obesity, diabetes, and genetic disorders). Optionally, the BTK-related disease is a neurodegenerative disorder. Optionally, the neurodegenerative disorder is Parkinson's disease. Optionally, the neurodegenerative disorder is Alexander disease, Alper's disease, Alzheimer disease, amyotrophic lateral sclerosis, ataxia telangiectasia, Batten disease (also known as Spielmeyer-Vogt-Sjogren-Batten disease), Canavan disease, Cockayne syndrome, Corticobasal degeneration, Creutzfeldt-Jakob disease, Huntington's disease, Kennedy's disease, Krabbe disease, Lewy body dementia, Machado-Joseph disease, Spinocerebellar ataxia type 3, multiple sclerosis, multiple system atrophy, Pelizaeus-Merzbacher disease, Pick's disease, Primary lateral sclerosis, Refsum's disease, Sandhoff disease, Schilder's disease, Spielmeyer-Vogt-Sjogren-Batten disease (also known as Batten disease), Spinocerebellar ataxia (multiple types with varying characteristics), Spinal muscular atrophy, Steele-Richardson-Olszewski disease, Tay-Sachs, Transmissible spongiform encephalopathies (TSE), or Tabes dorsalis.

Optionally, the BTK-related disease is an inflammatory disease. Generally, inflammatory disorders include, but are not limited to, respiratory or pulmonary disorders (including asthma, COPD, chronic bronchitis and cystic fibrosis); cardiovascular related disorders (including atherosclerosis, post-angioplasty, restenosis, coronary artery diseases and angina); inflammatory diseases of the joints (including rheumatoid and osteoarthritis); skin disorders (including dermatitis, eczematous dermatitis and psoriasis); post transplantation late and chronic solid organ rejection; multiple sclerosis; autoimmune conditions (including systemic lupus erythematosus, dermatomyositis, polymyositis, Sjogren's syndrome, polymyalgia rheumatica, temporal arteritis, Behcet's disease, Guillain Barre, Wegener's granulomatosus, polyarteritis nodosa); inflammatory neuropathies (including inflammatory polyneuropathies); vasculitis (including Churg-Strauss syndrome, Takayasu's arteritis); inflammatory disorders of adipose tissue; and proliferative disorders (including Kaposi's sarcoma and other proliferative disorders of smooth muscle cells).

Optionally, the BTK-related disease is ischemia, a gastrointestinal disorder, a viral infection (e.g., human immunodeficiency virus (HIV), including HIV type 1 (HIV-1) and HIV type 2 (HIV-2)), a bacterial infection, a central nervous system disorder, a spinal cord injury, or peripheral neuropathy.

The methods of treating or preventing a BTK-related disease (e.g., cancer) in a subject can further comprise administering to the subject one or more additional agents. The one or more additional agents and the compounds described herein or pharmaceutically acceptable salts or prodrugs thereof can be administered in any order, including concomitant, simultaneous, or sequential administration. Sequential administration can be administration in a temporally spaced order of up to several days apart. The methods can also include more than a single administration of the one or more additional agents and/or the compounds described herein or pharmaceutically acceptable salts or prodrugs thereof. The administration of the one or more additional agents and the compounds described herein or pharmaceutically acceptable salts or prodrugs thereof can be by the same or different routes and concurrently or sequentially.

Additional therapeutic agents include, but are not limited to, chemotherapeutic agents, anti-depressants, anxiolytics, antibodies, antivirals, steroidal and non-steroidal anti-inflammatories, conventional immunotherapeutic agents, cytokines, chemokines, and/or growth factors. The additional therapeutic agents can be biomolecules.

A chemotherapeutic agent is a compound or composition effective in inhibiting or arresting the growth of an abnormally growing cell. Thus, such an agent may be used therapeutically to treat cancer as well as other diseases marked by abnormal cell growth. Illustrative examples of chemotherapeutic compounds include, but are not limited to, bexarotene, gefitinib, erlotinib, gemcitabine, paclitaxel, docetaxel, topotecan, irinotecan, temozolomide, carmustine, vinorelbine, capecitabine, leucovorin, oxaliplatin, bevacizumab, cetuximab, panitumumab, bortezomib, oblimersen, hexamethylmelamine, ifosfamide, CPT-11, deflunomide, cycloheximide, dicarbazine, asparaginase, mitotant, vinblastine sulfate, carboplatin, colchicine, etoposide, melphalan, 6-mercaptopurine, teniposide, vinblastine, antibiotic derivatives (e.g. anthracyclines such as doxorubicin, liposomal doxorubicin, and diethylstilbestrol doxorubicin, bleomycin, daunorubicin, and dactinomycin); antiestrogens (e.g., tamoxifen); antimetabolites (e.g., fluorouracil (FU), 5-FU, methotrexate, floxuridine, interferon alpha-2B, glutamic acid, plicamycin, mercaptopurine, and 6-thioguanine); cytotoxic agents (e.g., carmustine, BCNU, lomustine, CCNU, cytosine arabinoside, cyclophosphamide, estramustine, hydroxyurea, procarbazine, mitomycin, busulfan, cisplatin, vincristine and vincristine sulfate); hormones (e.g., medroxyprogesterone, estramustine phosphate sodium, ethinyl estradiol, estradiol, megestrol acetate, methyltestosterone, diethylstilbestrol diphosphate, chlorotrianisene, and testolactone);

nitrogen mustard derivatives (e.g., mephalen, chlorambucil, mechlorethamine (nitrogen mustard) and thiotepa); and steroids (e.g., bethamethasone sodium phosphate).

Therapeutic agents further include, but are not limited to, levadopa, a dopamine agonist, an anticholinergic agent, a monoamine oxidase inhibitor, a COMT inhibitor, amantadine, rivastigmine, an NMDA antagonist, a cholinesterase inhibitor, riluzole, an anti-psychotic agent, an antidepressant, and tetrabenazine.

Any of the aforementioned therapeutic agents can be used in any combination with the compositions described herein. Combinations are administered either concomitantly (e.g., as an admixture), separately but simultaneously (e.g., via separate intravenous lines into the same subject), or sequentially (e.g., one of the compounds or agents is given first followed by the second). Thus, the term combination is used to refer to concomitant, simultaneous, or sequential administration of two or more agents.

Optionally, a compound or therapeutic agent as described herein may be administered in combination with a radiation therapy, an immunotherapy, a gene therapy, or a surgery.

The methods and compounds as described herein are useful for both prophylactic and therapeutic treatment. For prophylactic use, a therapeutically effective amount of the compounds and compositions or pharmaceutically acceptable salts thereof as described herein are administered to a subject prior to onset (e.g., before obvious signs of a BTK-related disease), during early onset (e.g., upon initial signs and symptoms of a BTK-related disease), or after the development of a BTK-related disease. Prophylactic administration can occur for several days to years prior to the manifestation of symptoms of a BTK-related disease. Therapeutic treatment involves administering to a subject a therapeutically effective amount of the compounds and compositions or pharmaceutically acceptable salts thereof as described herein after a BTK-related disease is diagnosed.

The compounds described herein are also useful in modulating BTK in a cell. Optionally, the compounds and compositions described herein are useful for inducing BTK degradation in a cell. The methods for inducing BTK degradation in a cell includes contacting a cell with an effective amount of one or more of the compounds as described herein. Optionally, the contacting is performed in vivo. Optionally, the contacting is performed in vitro.

The methods herein for prophylactic and therapeutic treatment optionally comprise selecting a subject with or at risk of developing a BTK-related disease. A skilled artisan can make such a determination using, for example, a variety of prognostic and diagnostic methods, including, for example, a personal or family history of the disease or condition, clinical tests (e.g., imaging, biopsy, genetic tests), and the like. Optionally, the methods herein can be used for preventing relapse of cancer in a subject in remission (e.g., a subject that previously had cancer).

### V. Kits

Also provided herein are kits for treating or preventing a BTK-related disease (e.g., cancer, a neurodegenerative disorder, an inflammatory diseases, and/or a metabolic disorder) in a subject. A kit can include any of the compounds or compositions described herein. A kit can further include one or more additional agents, such as one or more chemotherapeutic agents. A kit can include an oral formulation of any of the compounds or compositions described herein. A kit can include an intravenous formulation of any of the compounds or compositions described herein. A kit can additionally include directions for use of the kit (e.g., instructions for treating a subject), a container, a means for administering the compounds or compositions (e.g., a syringe), and/or a carrier.

As used herein the terms treatment, treat, or treating refer to a method of reducing one or more symptoms of a disease or condition. Thus in the disclosed method, treatment can refer to a 10%, 20%, 30%, 40%, 50%, 60%, 70%, 80%, 90%, or 100% reduction in the severity of one or more symptoms of the disease or condition. For example, a method for treating a disease is considered to be a treatment if there is a 10% reduction in one or more symptoms or signs (e.g., size of the tumor or rate of tumor growth) of the disease in a subject as compared to a control. As used herein, control refers to the untreated condition (e.g., the tumor cells not treated with the compounds and compositions described herein). Thus the reduction can be a 10%, 20%, 30%, 40%, 50%, 60%, 70%, 80%, 90%, 100%, or any percent reduction in between 10% and 100% as compared to native or control levels. It is understood that treatment does not necessarily refer to a cure or complete ablation of the disease, condition, or symptoms of the disease or condition.

As used herein, the terms prevent, preventing, and prevention of a disease or disorder refer to an action, for example, administration of a composition or therapeutic agent, that occurs before or at about the same time a subject begins to show one or more symptoms of the disease or disorder, which inhibits or delays onset or severity of one or more symptoms of the disease or disorder.

As used herein, references to decreasing, reducing, or inhibiting include a change of 10%, 20%, 30%, 40%, 50%, 60%, 70%, 80%, 90% or greater as compared to a control level. Such terms can include, but do not necessarily include, complete elimination.

As used herein, subject means both mammals and non-mammals. Mammals include, for example, humans; non-human primates, e.g., apes and monkeys; cattle; horses; sheep; rats; mice; pigs; and goats. Non-mammals include, for example, fish and birds.

Throughout this application, various publications are referenced. The disclosures of these publications in their entireties are hereby incorporated by reference into this application.

The examples below are intended to further illustrate certain aspects of the methods and compositions described herein, and are not intended to limit the scope of the claims.

### EXAMPLES

### Example 1: Compound Synthesis and Characterization

Materials: All chemicals were purchased from Sigma-Aldrich, Combi-blocks or Alfa Aesar, unless otherwise specified. All solvents and reagents were used as obtained without further purification.

Instrumentation: ¹H NMR and ¹³C NMR spectra were on a Varian (Palo Alto, CA) 400-MR spectrometer. Chemical shifts (8) are reported in ppm, and coupling constants (J) are in Hertz (Hz). The following abbreviations were used to explain the multiplicities: s=singlet, d=doublet, t=triplet, q=quartet, m=multiplet, br=broad. Flash chromatography was performed on a Teledyne ISCO CombiFlash Rf 200. ESI mass spectrometry was measured on an Agilent Mass Spectrometer.

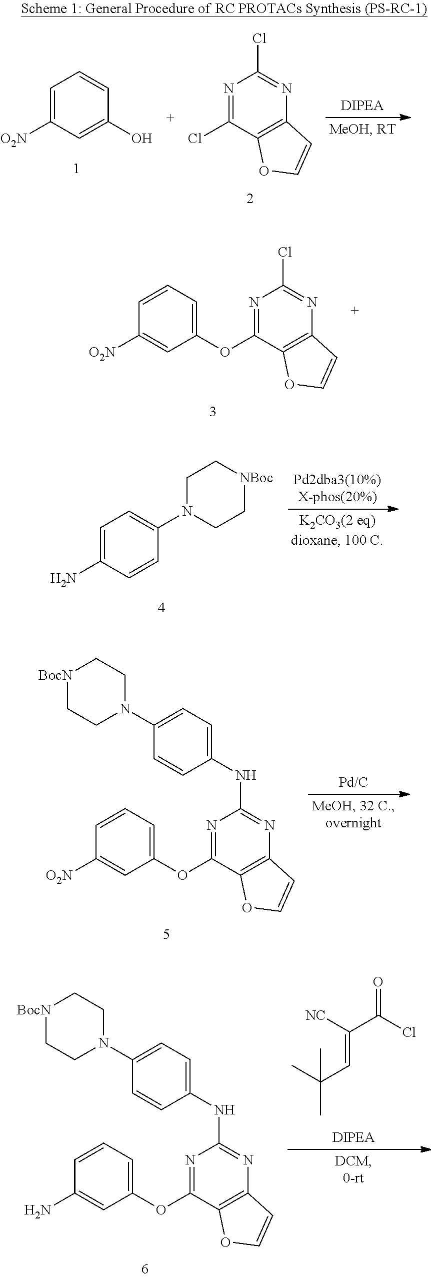

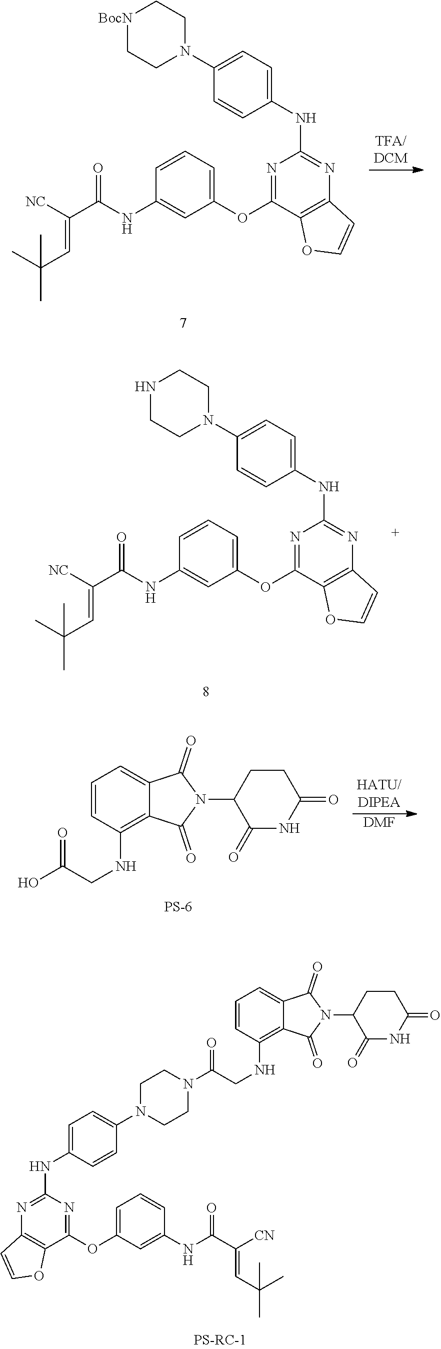

### 2-Chloro-4-(3-nitrophenoxy)furo[3,2-d]pyrimidine (3)

To a 250 mL of Schlenk tube equipped with a magnetic stir bar were added compound 1 (3 g, 21.6 mmol), 2 (4 g, 21.6 mmol) and DIPEA (6.5 g, 50 mmol) in MeOH (300 mL). The mixture was stirred under air at room temperature overnight. Upon the completion of the reaction, the resulting solid was filtered and dried over under a reduced pressure to obtain the title compound 3 (3.7 g, 60%). ¹H NMR (400 MHZ, DMSO-d6) δ 8.63 (d, J=2.3 Hz, 1H), 8.34 (t, J=2.3 Hz, 1H), 8.23 (dd, J=8.0, 2.0 Hz, 1H), 7.96-7.87 (m, 1H), 7.82 (t, J=8.2 Hz, 1H), 7.29 (d, J=2.2 Hz, 1H).

### tert-Butyl 4-(4-((4-(3-nitrophenoxy)furo[3,2-d]pyrimidin-2-yl)amino)phenyl)piperazine-1-carboxylate (5)

To a 25 mL of Schlenk tube equipped with a magnetic stir bar were added compound 3 (1.5 g, 5 mmol), 4 (1.4 g, 5 mmol), K₂CO₃ (1.4 g, 10 mmol), Pd₂(dba)₃ (456 mg, 10 mol %) and X-phos (457 mg, 20 mol %). Then dioxane (20 mL) was added under N₂. The Schlenk tube was screw capped and heated to 100° C. for 12 hours. Then the reaction mixture was cooled to room temperature, and sat. NH₄Cl aq. was poured into the reaction mixture and extracted with EtOAc. The organic layer was washed with brine, dried over Na₂SO₄, and filtered. The filtrate was concentrated in vacuo and the residue was purified by flash column chromatography to afford 5 (1.9 g, 73%) as a yellow solid. MS (EI): m/z 533.2 [M+H]+.

### tert-Butyl 4-(4-((4-(3-aminophenoxy)furo[3,2-d]pyrimidin-2-yl)amino)phenyl)piperazine-1-carboxylate (6)

To a flask was added compound 5 (1.06 g, 2 mmol) and Pd/C (110 mg, 10%) in MeOH (50 mL). The mixture was stirred under 1 atm H₂ at room temperature overnight. LC-MS showed compound 5 converted into compound 6 completely. Then the reaction mixture was filtered, and the filtrate was concentrated in vacuo. The mixture was extracted with EtOAc and washed with brine, dried over Na₂SO₄, and filtered. The filtrate was concentrated in vacuo and the residue was purified by flash column chromatography to afford 6 (1.9 g, 90%) as a white solid. ¹H NMR (400 MHz, DMSO-d6) δ 9.12 (s, 1H), 8.26 (d, J=2.2 Hz, 1H), 7.48 (d, J=8.7 Hz, 2H), 7.10 (t, J=8.0 Hz, 1H), 6.92 (d, J=2.2 Hz, 1H), 6.78 (d, J=9.1 Hz, 2H), 6.51 (dd, J=7.9, 2.1 Hz, 1H), 6.45 (t, J=2.2 Hz, 1H), 6.41 (dd, J=7.9, 2.3 Hz, 1H), 5.32 (s, 2H), 3.49-3.39 (m, 4H), 2.95 (t, J=5.1 Hz, 4H), 1.42 (s, 9H). MS (EI): m/z 503.2 [M+H].

### tert-Butyl (E)-4-(4-((4-(3-(2-cyano-4,4-dimethylpent-2-enamido)phenoxy)furo[3,2-d]pyrimidin-2-yl)amino)phenyl)piperazine-1-carboxylate (7)

To compound 6 (500 mg, 1 mmol) was added compound (E)-2-cyano-4,4-dimethylpent-2-enoyl chloride (257 mg, 1.5 mol) and DIPEA (387 mg, 3 mmol) in DCM (20 mL). The mixture was stirred at room temperature for 30 minutes. LC-MS showed compound 6 converted into compound 7 completely. Then the reaction mixture was concentrated in vacuo to give compound 7. MS (EI): m/z 638.2 [M+H]+.

### (E)-2-cyano-N-(3-((2-((4-(4-((2-(2,6-dioxopiperidin-3-yl)-1,3-dioxoisoindolin-4-yl)glycyl)piperazin-1-yl)phenyl)amino)furo[3,2-d]pyrimidin-4-yl)oxy)phenyl)-4,4-dimethylpent-2-enamide (PS-RC-1)

In a 25 mL flask was added 7 (32 mg, 0.05 mmol) in TFA/DCM (5 mL, 1/1). The mixture was stirred for 30 minutes at room temperature. Then the solvent was removed in vacuo to give the deprotected intermediate, which was used for next step without further purification. To the above intermediate was added compound PS-6 (33 mg, 0.1 mol), HATU (38 mg, 0.1 mmol) and DIPEA (32 mg, 0.25 mmol) in DMF (2 mL). The mixture was stirred at room temperature for 30 minutes. Then the reaction mixture was concentrated in vacuo and the residue was purified by PrepHPLC with a reverse phase C18 column to afford the product as a yellow solid PS-RC-1 (13 mg, 30%). ¹H NMR (400 MHZ, DMSO-d6) δ 11.09 (s, 1H), 10.44 (s, 1H), 9.18 (s, 1H), 8.30 (d, J=2.2 Hz, 1H), 7.71-7.55 (m, 3H), 7.53-7.45 (m, 2H), 7.42 (d, J=8.4 Hz, 2H), 7.16-7.03 (m, 4H), 6.95 (d, J=2.2 Hz, 1H), 6.76 (d, J=8.5 Hz, 2H), 5.08 (dd, J=12.9, 5.5 Hz, 1H), 4.24 (d, J=4.5 Hz, 2H), 3.71-3.54 (m, 4H), 3.12-2.97 (m, 4H), 2.89 (td, J=17.6, 15.6, 5.4 Hz, 1H), 2.67-2.52 (m, 2H), 2.04 (d, J=12.1 Hz, 1H), 1.27 (s, 9H). ¹³C NMR (100 MHZ, DMSO-d6) δ 173.3, 170.5, 169.3, 167.8, 167.1, 166.8, 161.2, 156.7, 156.6, 153.4, 153.1, 152.6, 145.9, 145.8, 139.9, 136.6, 134.1, 132.5, 130.4, 129.0, 120.0, 1187, 118.2, 116.9, 115.5, 114.4, 111.3, 110.0, 109.6, 107.4, 50.0, 49.7, 49.0, 44.2, 44.1, 41.9, 34.9, 31.4, 29.0, 22.6. HRMS (m/z): [M+H]+ calcd. for C₄₅H₄₃N₁₀O₈, 851.3265; found: 851.3267.

(E)-2-cyano-4,4-dimethyl-N-(3-((2-((4-(4-((2-(1-methyl-2,6-dioxopiperidin-3-yl)-1,3-dioxoisoindolin-4-yl)glycyl)piperazin-1-yl)phenyl)amino)furo[3,2-d]pyrimidin-4-yl)oxy)phenyl)pent-2-enamide (PS-RC-1-Me)

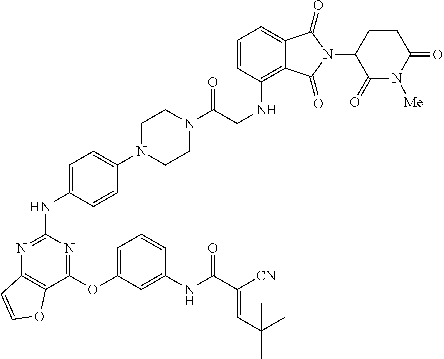 [SMILES: CN1C(=O)CCC(N2C(=O)c3cccc(NCC(=O)N4CCN(c5ccc(Nc6nc(Oc7cccc(NC(=O)/C(C#N)=C/C(C)(C)C)c7)c7occc7n6)cc5)CC4)c3C2=O)C1=O]

The general procedure outlined above for synthesizing PS-RC-1 was modified and used to prepare PS-RC-1-Me.

¹H NMR (400 MHZ, DMSO-d6) δ 10.43 (s, 1H), 9.18 (s, 1H), 8.30 (d, J=2.2 Hz, 1H), 7.67-7.53 (m, 3H), 7.52-7.45 (m, 2H), 7.42 (d, J=8.6 Hz, 2H), 7.17-7.01 (m, 4H), 6.95 (d, J=2.2 Hz, 1H), 6.77 (d, J=8.3 Hz, 2H), 5.14 (dd, J=13.0, 5.4 Hz, 1H), 4.25 (d, J=4.5 Hz, 2H), 3.65 (d, J=10.2 Hz, 4H), 3.07 (s, 2H), 3.03 (s, 3H), 3.00-2.90 (m, 2H), 2.82-2.72 (m, 1H), 2.62-2.50 (m, 2H), 2.06 (d, J=10.3 Hz, 1H), 1.27 (s, 9H). ¹³C NMR (100 MHZ, DMSO-d6) δ 172.3, 170.3, 169.2, 167.8, 167.0, 166.8, 164.9, 161.2, 156.7, 156.6, 153.4, 153.1, 152.8, 152.6, 145.9, 145.7, 139.9, 136.7, 134.1, 132.4, 130.4, 129.0, 120.0, 118.7, 118.2, 116.9, 115.5, 114.4, 111.3, 110.0, 107.4, 50.0, 49.7, 49.6, 44.1, 41.9, 34.9, 31.6, 29.3, 29.0, 27.01, 23.6. HRMS (m/z): [M+H]+ calcd. for C₄₆H₄₅N₁₀O₈, 865.3422; found: 865.3412.

(E)-2-cyano-N-(3-((2-((4-(4-(3-(2-(2-((2-(2,6-dioxopiperidin-3-yl)-1,3-dioxoisoindolin-4-yl)amino)acetamido)ethoxy)propanoyl)piperazin-1-yl)phenyl)amino)furo[3,2-d]pyrimidin-4-yl)oxy)phenyl)-4,4-dimethylpent-2-enamide. (PS-RC-2)

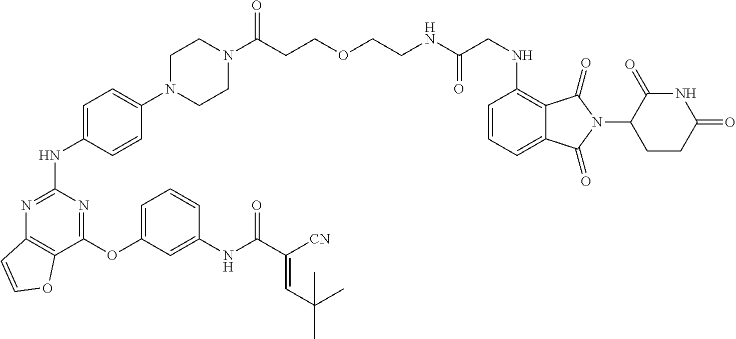 [SMILES: CC(C)(C)/C=C(\C#N)C(=O)Nc1cccc(Oc2nc(Nc3ccc(N4CCN(C(=O)CCOCCNC(=O)CNc5cccc6c5C(=O)N(C5CCC(=O)NC5=O)C6=O)CC4)cc3)nc3ccoc23)c1]

The general procedure outlined above for synthesizing PS-RC-1 was modified and used to prepare PS-RC-2.

¹H NMR (400 MHZ, DMSO-d6) δ 9.13 (d, J=3.9 Hz, 1H), 8.27 (d, J=2.2 Hz, 1H), 8.14 (t, J=5.6 Hz, 1H), 7.64-7.51 (m, 3H), 7.50-7.39 (m, 2H), 7.37 (d, J=8.3 Hz, 2H), 7.06 (dd, J=11.9, 7.1 Hz, 2H), 6.92 (t, J=4.1 Hz, 2H), 6.83 (d, J=8.5 Hz, 1H), 6.70 (d, J=8.5 Hz, 2H), 5.04 (dd, J=12.8, 5.4 Hz, 1H), 3.91 (d, J=5.6 Hz, 2H), 3.60 (t, J=6.4 Hz, 2H), 3.54 (s, 4H), 3.39 (t, J=5.8 Hz, 2H), 3.23 (d, J=5.7 Hz, 2H), 2.99-2.88 (m, 4H), 2.88-2.79 (m, 1H), 2.62-2.50 (m, 4H), 1.99 (d, J=11.9 Hz, 1H), 1.24 (s, 5H), 1.01-0.89 (m, 4H). ¹³C NMR (100 MHz, DMSO-d6) δ 173.3, 170.5, 169.2, 169.1, 169.0, 167.8, 161.3, 156.7, 156.6, 153.4, 153.1, 152.8, 146.3, 145.9, 139.9, 136.7, 134.0, 132.5, 130.6, 129.0, 120.0, 117.9, 116.8, 111.4, 110.3, 107.4, 69.1, 66.9, 50.3, 49.8, 49.0, 45.6, 45.3, 43.2, 33.2, 31.4, 29.3, 29.1, 23.6, 22.6. MS (EI): m/z 966.3 [M+H]+.

(E)-2-cyano-N-(3-((2-((4-(4-(3-(2-(2-(2-((2-(2,6-dioxopiperidin-3-yl)-1,3-dioxoisoindolin-4-yl)amino)acetamido)ethoxy)ethoxy)propanoyl)piperazin-1-yl)phenyl)amino)furo[3,2-d]pyrimidin-4-yl)oxy)phenyl)-4,4-dimethylpent-2-enamide (PS-RC-3)

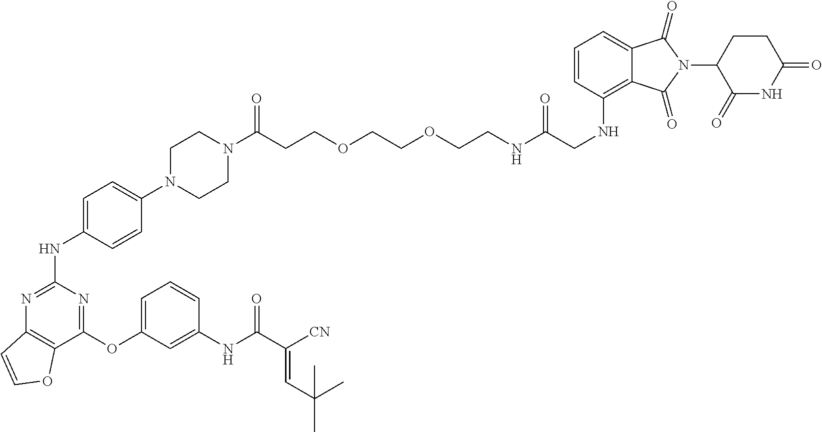 [SMILES: CC(C)(C)/C=C(\C#N)C(=O)Nc1cccc(Oc2nc(Nc3ccc(N4CCN(C(=O)CCOCCOCCNC(=O)CNc5cccc6c5C(=O)N(C5CCC(=O)NC5=O)C6=O)CC4)cc3)nc3ccoc23)c1]

The general procedure outlined above for synthesizing PS-RC-1 was modified and used to prepare PS-RC-3.

¹H NMR (400 MHZ, DMSO-d6) δ 9.15 (d, J=3.8 Hz, 1H), 8.30 (t, J=1.8 Hz, 1H), 8.17 (t, J=5.7 Hz, 1H), 7.59 (td, J=14.6, 13.7, 8.3 Hz, 3H), 7.53-7.42 (m, 2H), 7.40 (d, J=8.4 Hz, 2H), 7.08 (dd, J=14.4, 7.5 Hz, 2H), 6.94 (d, J=1.9 Hz, 2H), 6.85 (d, J=8.4 Hz, 1H), 6.72 (d, J=8.5 Hz, 2H), 5.06 (dd, J=12.9, 5.4 Hz, 1H), 3.93 (d, J=5.5 Hz, 2H), 3.63 (t, J=6.7 Hz, 2H), 3.56 (d, J=5.2 Hz, 4H), 3.48 (d, J=3.5 Hz, 4H), 3.40 (d, J=5.8 Hz, 2H), 3.25 (d, J=6.0 Hz, 2H), 3.02-2.90 (m, 4H), 2.88-2.81 (m, 1H), 2.65-2.51 (m, 4H), 2.01 (d, J=11.8 Hz, 1H), 1.26 (d, J=1.3 Hz, 5H), 1.04-0.90 (m, 4H). ¹³C NMR (100 MHz, DMSO-d6) δ 173.3, 170.5, 169.2, 169.1, 169.0, 167.8, 164.9, 156.7, 156.6, 153.4, 153.1, 152.8, 152.7, 152.6, 146.2, 145.9, 139.9, 136.6, 134.0, 132.5, 130.6, 130.2, 129.0, 120.0, 119.9, 117.9, 116.8, 111.4, 110.3, 107.4, 70.0, 69.4, 67.2, 50.3, 49.7, 49.0, 45.6, 45.4, 43.2, 39.1, 33.2, 31.4, 29.3, 29.1, 23.6, 22.6. MS (EI): m/z 1010.4 [M+H]+.

(E)-2-cyano-N-(3-((2-((4-(4-(1-((2-(2,6-dioxopiperidin-3-yl)-1,3-dioxoisoindolin-4-yl)amino)-2-oxo-6,9,12-trioxa-3-azapentadecan-15-oyl)piperazin-1-yl)phenyl)amino)furo[3,2-d]pyrimidin-4-yl)oxy)phenyl)-4,4-dimethylpent-2-enamide (PS-RC-4)

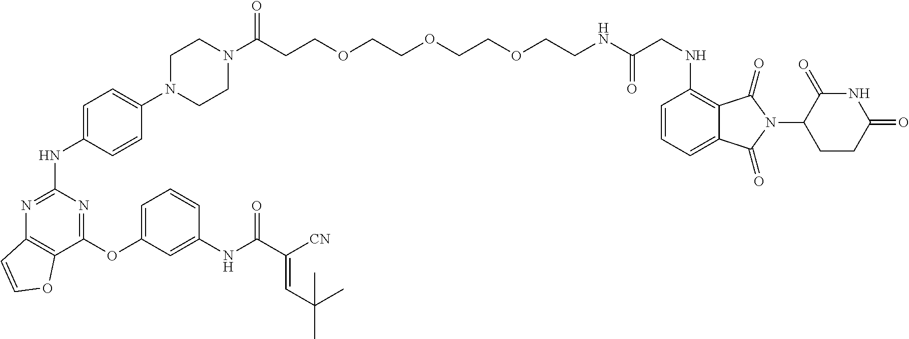 [SMILES: CC(C)(C)/C=C(\C#N)C(=O)Nc1cccc(Oc2nc(Nc3ccc(N4CCN(C(=O)CCOCCOCCOCCNC(=O)CNc5cccc6c5C(=O)N(C5CCC(=O)NC5=O)C6=O)CC4)cc3)nc3ccoc23)c1]

The general procedure outlined above for synthesizing PS-RC-1 was modified and used to prepare PS-RC-4.

¹H NMR (400 MHZ, DMSO-d6) δ 9.16 (s, 1H), 8.30 (d, J=2.2 Hz, 1H), 8.16 (t, J=5.7 Hz, 1H), 7.65-7.52 (m, 3H), 7.47 (d, J=7.3 Hz, 2H), 7.40 (d, J=8.7 Hz, 2H), 7.13-7.03 (m, 2H), 6.94 (t, J=3.5 Hz, 2H), 6.85 (d, J=8.6 Hz, 1H), 6.73 (d, J=8.4 Hz, 2H), 5.07 (dd, J=12.9, 5.4 Hz, 1H), 3.93 (d, J=5.6 Hz, 2H), 3.63 (t, J=6.6 Hz, 2H), 3.56 (d, J=5.5 Hz, 4H), 3.48 (d, J=6.1 Hz, 8H), 3.41 (t, J=5.7 Hz, 2H), 3.24 (t, J=5.9 Hz, 2H), 2.96 (d, J=23.0 Hz, 4H), 2.90-2.81 (m, 1H), 2.64-2.52 (m, 4H), 2.02 (d, J=11.5 Hz, 1H), 1.27 (s, 5H), 0.98 (d, J=27.6 Hz, 4H). ¹³C NMR (100 MHz, DMSO-d6) δ 173.3, 170.5, 169.2, 169.1, 169.0, 167.8, 153.4, 153.1, 152.8, 152.6, 146.3, 145.9, 136.6, 134.0, 132.5, 120.0, 119.9, 117.9, 116.9, 116.8, 111.4, 110.3, 107.4, 70.21, 70.16, 70.1, 70.0, 69.4, 67.2, 50.3, 49.7, 49.0, 45.6, 45.4, 43.2, 39.1, 33.2, 31.4, 29.3, 23.6, 22.6. MS (EI): m/z 1054.4 [M+H]+.

### N-(3-((2-((4-(4-((2-(2,6-dioxopiperidin-3-yl)-1,3-dioxoisoindolin-4-yl)glycyl)piperazin-1-yl)phenyl)amino)furo[3,2-d]pyrimidin-4-yl)oxy)phenyl)acrylamide (PS-IRC-1)

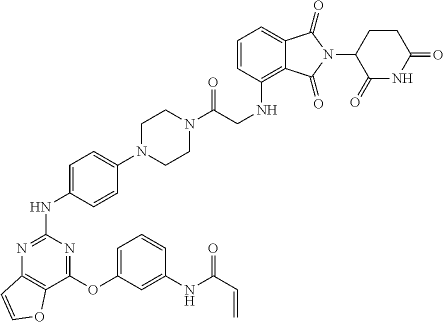 [SMILES: C=CC(=O)Nc1cccc(Oc2nc(Nc3ccc(N4CCN(C(=O)CNc5cccc6c5C(=O)N(C5CCC(=O)NC5=O)C6=O)CC4)cc3)nc3ccoc23)c1]

The general procedure outlined above for synthesizing PS-RC-1 was modified and used to prepare PS-IRC-1.

¹H NMR (400 MHZ, DMSO-d6) δ 11.11 (s, 1H), 10.37 (s, 1H), 9.17 (s, 1H), 8.30 (d, J=2.2 Hz, 1H), 7.73-7.59 (m, 3H), 7.51-7.34 (m, 3H), 7.17-7.03 (m, 4H), 6.95 (d, J=2.2 Hz, 1H), 6.75 (d, J=8.7 Hz, 2H), 6.44 (dd, J=17.0, 10.1 Hz, 1H), 6.27 (dd, J=17.0, 2.1 Hz, 1H), 5.78 (dd, J=10.0, 2.1 Hz, 1H), 5.08 (dd, J=12.9, 5.4 Hz, 1H), 4.25 (d, J=4.5 Hz, 2H), 3.65 (dd, J=11.4, 5.9 Hz, 4H), 3.11-2.96 (m, 4H), 2.89 (td, J=17.0, 15.3, 5.4 Hz, 1H), 2.65-2.51 (m, 2H), 2.12-2.00 (m, 1H). ¹³C NMR (100 MHz, DMSO-d6) δ 173.3, 170.5, 169.2, 167.8, 166.8, 163.8, 156.7, 156.6, 153.4, 153.1, 152.7, 145.9, 145.8, 140.8, 136.6, 134.1, 132.5, 132.1, 130.4, 129.0, 127.8, 120.0, 118.7, 117.2, 117.0, 116.9, 113.3, 111.3, 110.0, 107.4, 50.0, 49.7, 49.0, 44.1, 41.9, 31.4, 22.6. HRMS (m/z): [M+H]+ calcd. for C₄₀H₃₆N₉O₈, 770.2687; found: 770.2690.

### N-(3-((2-((4-(4-((2-(2,6-dioxopiperidin-3-yl)-1,3-dioxoisoindolin-4-yl)glycyl)piperazin-1-yl)phenyl)amino)furo[3,2-d]pyrimidin-4-yl)oxy)phenyl)propionamide (PS-RNC-1)

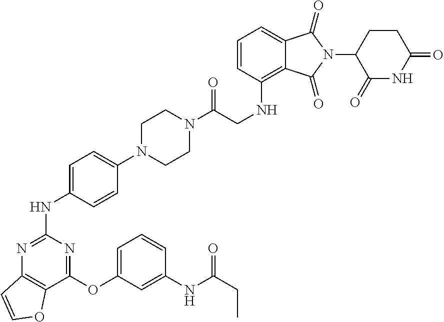 [SMILES: CCC(=O)Nc1cccc(Oc2nc(Nc3ccc(N4CCN(C(=O)CNc5cccc6c5C(=O)N(C5CCC(=O)NC5=O)C6=O)CC4)cc3)nc3ccoc23)c1]

The general procedure outlined above for synthesizing PS-RC-1 was modified and used to prepare PS-RNC-1.

¹H NMR (400 MHZ, DMSO-d6) δ 11.11 (s, 1H), 10.08 (s, 1H), 9.15 (s, 1H), 8.30 (d, J=2.2 Hz, 1H), 7.69-7.57 (m, 2H), 7.55 (d, J=8.2 Hz, 1H), 7.41 (t, J=8.2 Hz, 3H), 7.19-7.06 (m, 3H), 7.04-6.99 (m, 1H), 6.95 (d, J=2.1 Hz, 1H), 6.77 (d, J=8.7 Hz, 2H), 5.08 (dd, J=12.9, 5.4 Hz, 1H), 4.25 (d, J=4.5 Hz, 2H), 3.65 (d, J=10.9 Hz, 4H), 3.05 (dd, J=25.9, 6.1 Hz, 4H), 2.89 (ddd, J=17.3, 13.9, 5.5 Hz, 1H), 2.66-2.52 (m, 2H), 2.33 (q, J=7.5 Hz, 2H), 2.12-1.96 (m, 1H), 1.07 (t, J=7.5 Hz, 3H). ¹³C NMR (100 MHz, DMSO-d6) δ 173.3, 172.7, 170.5, 169.2, 167.8, 166.8, 156.7, 156.5, 153.4, 153.0, 152.8, 145.9, 145.8, 141.2, 136.6, 134.2, 132.5, 130.3, 129.0, 120.0, 118.7, 116.9, 116.7, 116.6, 112.8, 111.3, 110.0, 107.4, 50.03, 49.7, 49.0, 44.2, 44.1, 41.9, 31.4, 30.0, 22.6, 10.0. HRMS (m/z): [M+H]+ calcd. for C₄₀H₃₈N₉O₈, 772.2843; found: 772.2831.

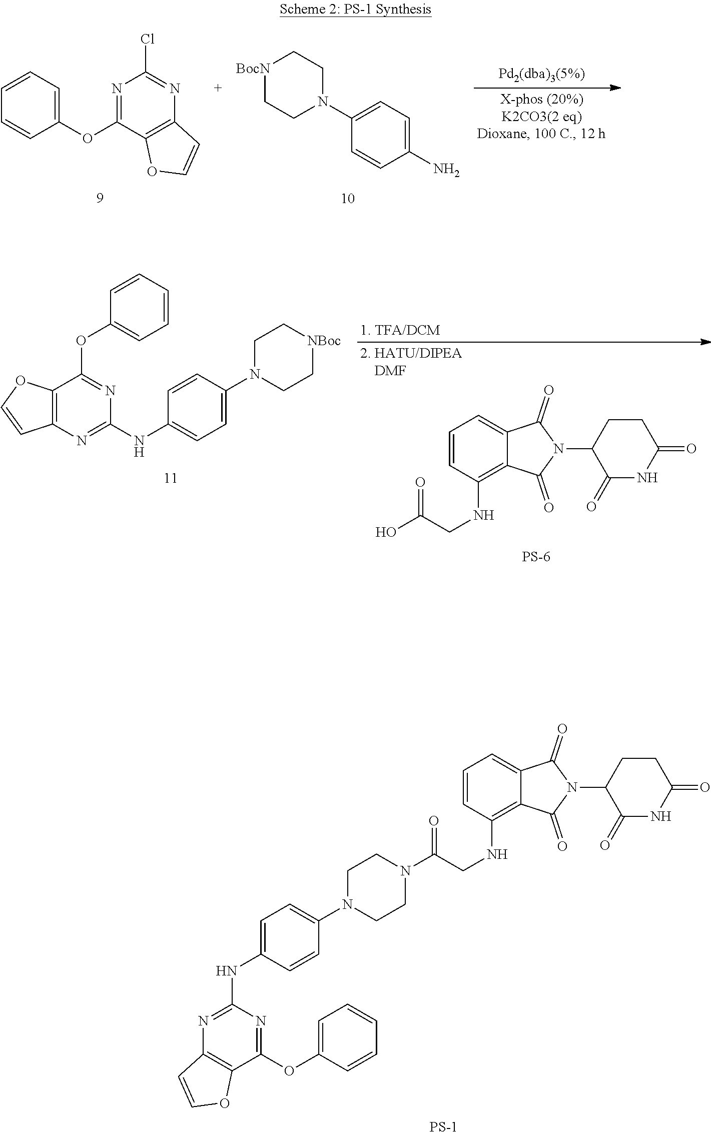

### tert-Butyl 4-(4-((4-phenoxyfuro[3,2-d]pyrimidin-2-yl)amino)phenyl)piperazine-1-carboxylate (11)

To a 25 mL of Schlenk tube equipped with a magnetic stir bar were added compound 9 (49 mg, 0.2 mmol), 10 (55 mg, 0.2 mmol), K₂CO₃ (55 mg, 0.4 mmol), Pd₂(dba)₃ (18 mg, 10 mol %) and X-phos (19 mg, 20 mol %). Then dioxane (5 mL) was added under N₂. The Schlenk tube was screw capped and heated to 100° C. for 12 hours. Then the reaction mixture was cooled to room temperature, and sat. NH₄Cl aq. was poured into the reaction mixture and extracted with EtOAc. The organic layer was washed with brine, dried over Na₂SO₄, and filtered. The filtrate was concentrated in vacuo and the residue was purified by flash column chromatography to afford 11 (63 mg, 65%) as a white solid. ¹H NMR (400 MHZ, DMSO-d6) δ 9.38-9.28 (m, 1H), 8.29 (dd, J=5.5, 1.9 Hz, 1H), 7.54-7.45 (m, 2H), 7.40-7.27 (m, 3H), 7.27-7.19 (m, 2H), 6.72 (d, J=8.5 Hz, 2H), 6.33 (dd, J=5.6, 1.9 Hz, 1H), 3.43 (d, J=5.4 Hz, 4H), 2.94 (t, J=5.2 Hz, 4H), 1.41 (d, J=2.0 Hz, 9H). MS (EI): m/z 488.2 [M+H]+.

### 2-(2,6-Dioxopiperidin-3-yl)-4-((2-oxo-2-(4-(4-((4-phenoxyfuro[3,2-d]pyrimidin-2-yl)amino)phenyl)piperazin-1-yl)ethyl)amino)isoindoline-1,3-dione (PS-1)

In a 25 mL flask was added 11 (27 mg, 0.05 mmol) in TFA/DCM (5 mL, 1/1). The mixture was stirred for 30 min at room temperature. Then the solvent was removed in vacuo to give the deprotected intermediate, which was used for next step without further purification. To the above intermediate was added compound PS-6 (33 mg, 0.1 mol), HATU (38 mg, 0.1 mmol) and DIPEA (32 mg, 0.25 mmol) in DMF (2 mL). The mixture was stirred at room temperature for 30 minutes. Then the reaction mixture was concentrated in vacuo and the residue was purified by PrepHPLC with a reverse phase C18 column to afford the product as a yellow solid PS-1 (14 mg, 40%). ¹H NMR (400 MHZ, DMSO-d6) δ 11.12 (s, 1H), 9.12 (s, 1H), 8.28 (d, J=2.2 Hz, 1H), 7.61 (t, J=7.8 Hz, 1H), 7.55-7.47 (m, 2H), 7.42 (d, J=8.5 Hz, 2H), 7.36 (d, J=7.6 Hz, 3H), 7.15-7.05 (m, 3H), 6.94 (d, J=2.2 Hz, 1H), 6.76 (d, J=8.8 Hz, 2H), 5.08 (dd, J=12.9, 5.4 Hz, 1H), 4.24 (d, J=4.6 Hz, 2H), 3.65 (dt, J=12.2, 4.9 Hz, 4H), 3.04 (dt, J=25.5, 5.1 Hz, 4H), 2.89 (ddd, J=17.2, 14.0, 5.5 Hz, 1H), 2.65-2.52 (m, 2H), 2.10-1.98 (m, 1H). ¹³C NMR (100 MHz, DMSO-d6) δ 173.3, 170.5, 169.3, 167.8f, 166.8, 156.7, 156.4, 153.5, 152.9, 152.5, 145.9, 145.8, 136.6, 134.2, 132.5, 130.2, 129.1, 126.1, 122.4, 120.0, 118.7, 117.0, 111.3, 110.0, 107.4, 50.0, 49.8, 49.0, 44.2, 44.1, 42.0, 31.4, 22.6. HRMS (m/z): [M+H]+ calcd. for C₃₇H₃₃N₈O₇, 701.2472; found: 701.2459.

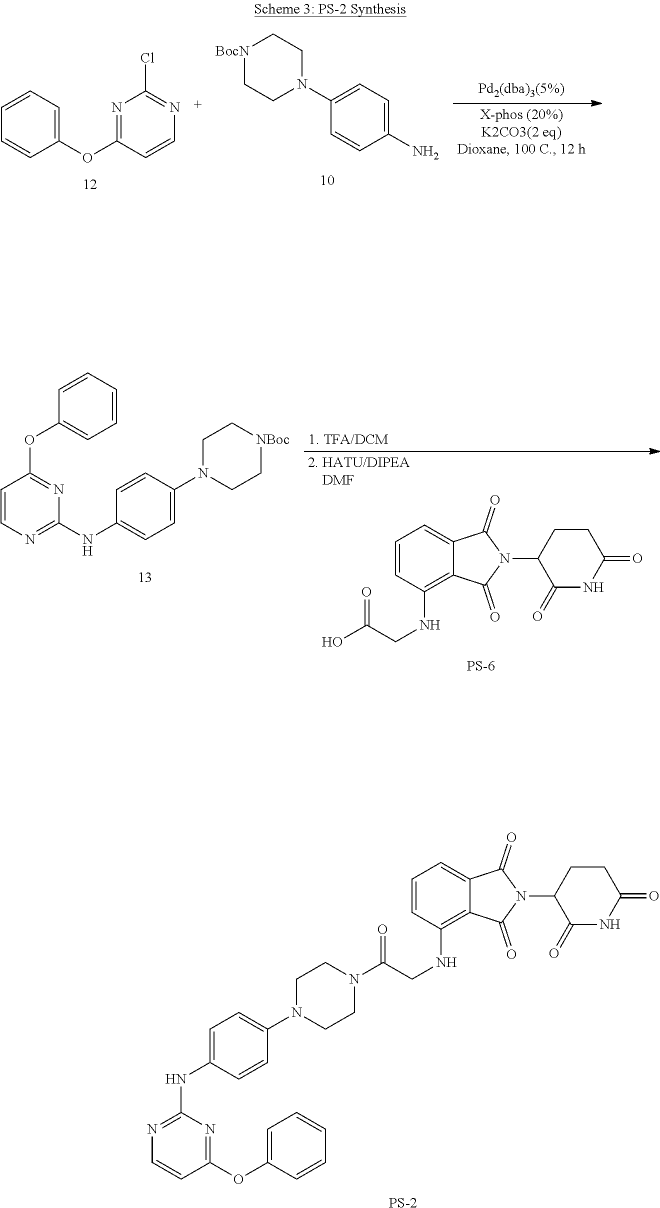

### tert-Butyl 4-(4-((4-phenoxypyrimidin-2-yl)amino)phenyl)piperazine-1-carboxylate (13)

To a 25 mL of Schlenk tube equipped with a magnetic stir bar were added compound 12 (41 mg, 0.2 mmol), 10 (55 mg, 0.2 mmol), K₂CO₃ (55 mg, 0.4 mmol), Pd₂(dba)₃ (18 mg, 10 mol %) and X-phos (19 mg, 20 mol %). Then dioxane (5 mL) was added under N₂. The Schlenk tube was screw capped and heated to 100° C. for 12 hours. Then the reaction mixture was cooled to room temperature, and sat. NH₄Cl aq. was poured into the reaction mixture and extracted with EtOAc. The organic layer was washed with brine, dried over Na₂SO₄, and filtered. The filtrate was concentrated in vacuo and the residue was purified by flash column chromatography to afford 13 (67 mg, 75%) as a white solid. ¹H NMR (400 MHZ, DMSO-d6) δ 9.11 (s, 1H), 8.28 (t, J=1.5 Hz, 1H), 7.54-7.48 (m, 2H), 7.44-7.31 (m, 5H), 6.94 (t, J=1.5 Hz, 1H), 6.72 (d, J=8.7 Hz, 2H), 3.44 (t, J=5.1 Hz, 4H), 2.94 (t, J=5.1 Hz, 4H), 1.42 (s, 9H). MS (EI): m/z 448.2 [M+H]+.

### 2-(2,6-Dioxopiperidin-3-yl)-4-((2-oxo-2-(4-(4-((4-phenoxypyrimidin-2-yl)amino)phenyl)piperazin-1-yl)ethyl)amino)isoindoline-1,3-dione (PS-2)

In a 25 mL flask was added 13 (22 mg, 0.05 mmol) in TFA/DCM (5 mL, 1/1). The mixture was stirred for 30 min at room temperature. Then the solvent was removed in vacuo to give the deprotected intermediate, which was used for next step without further purification. To the above intermediate was added compound PS-6 (33 mg, 0.1 mol), HATU (38 mg, 0.1 mmol) and DIPEA (32 mg, 0.25 mmol) in DMF (2 mL). The mixture was stirred at room temperature for 30 min. Then the reaction mixture was concentrated in vacuo and the residue was purified by PrepHPLC with a reverse phase C18 column to afford the product as a yellow solid PS-2 (11 mg, 34%). ¹H NMR (400 MHZ, DMSO-d6) δ 11.11 (s, 1H), 9.37 (s, 1H), 8.29 (dd, J=5.6, 1.6 Hz, 1H), 7.62 (dd, J=8.5, 7.0 Hz, 1H), 7.52-7.44 (m, 2H), 7.34 (dt, J=14.8, 7.9 Hz, 3H), 7.26-7.20 (m, 2H), 7.16-7.03 (m, 3H), 6.76 (d, J=8.5 Hz, 2H), 6.34 (dd, J=5.6, 1.6 Hz, 1H), 5.08 (dd, J=12.7, 5.2 Hz, 1H), 4.25 (d, J=4.2 Hz, 2H), 3.63 (d, J=9.7 Hz, 4H), 3.12-2.99 (m, 4H), 2.93-2.79 (m, 1H), 2.65-2.52 (m, 2H), 2.09-1.96 (m, 1H). ¹³C NMR (100 MHZ, DMSO-d6) δ 173.3, 170.5, 169.8, 169.2, 167.8, 166.8, 160.3, 160.2, 152.9, 145.9, 136.6, 133.4, 132.5, 130.2, 125.9, 122.4, 120.5, 118.7, 116.9, 111.3, 110.0, 49.9, 49.7, 49.0, 44.1, 41.9, 31.4, 22.6. HRMS (m/z): [M+H]+ calcd. for C₃₅H₃₃N₈O₆, 661.2523; found: 661.2514.

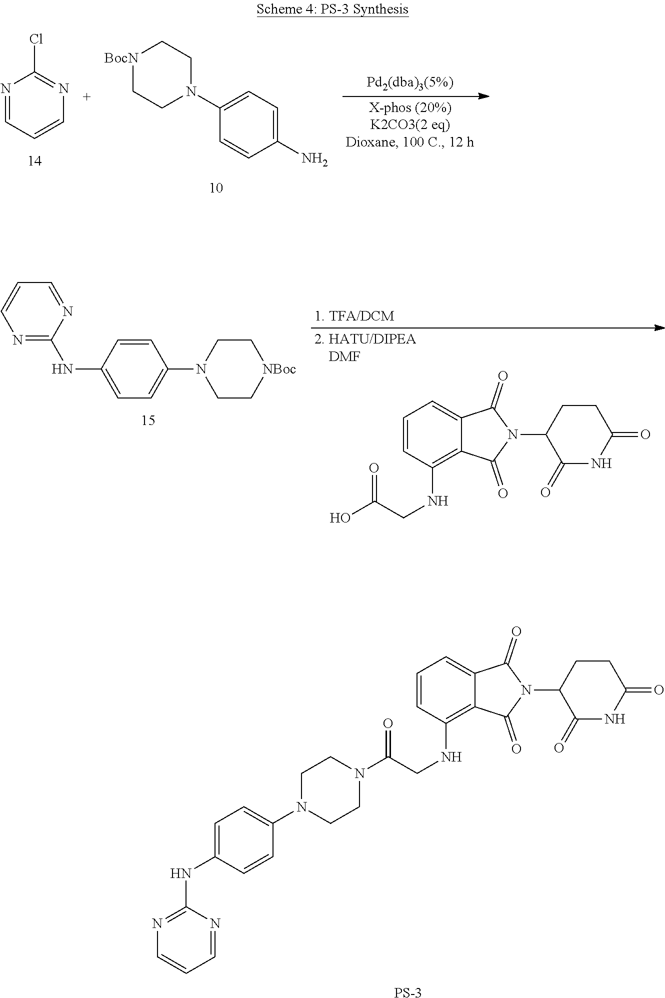 [SMILES: Clc1ncccn1 ; CC(C)(C)OC(=O)N1CCN(c2ccc(N)cc2)CC1 ; CC(C)(C)OC(=O)N1CCN(c2ccc(Nc3ncccn3)cc2)CC1 ; O=C(O)CNc1cccc2c1C(=O)N(C1CCC(=O)NC1=O)C2=O ; O=C1CCC(N2C(=O)c3cccc(NCC(=O)N4CCN(c5ccc(Nc6ncccn6)cc5)CC4)c3C2=O)C(=O)N1 ; "PS-3"]

### tert-Butyl 4-(4-(pyrimidin-2-ylamino)phenyl)piperazine-1-carboxylate (15)

To a 25 mL of Schlenk tube equipped with a magnetic stir bar were added compound 14 (23 mg, 0.2 mmol), 10 (55 mg, 0.2 mmol), K₂CO₃ (55 mg, 0.4 mmol), Pd₂(dba)₃ (18 mg, 10 mol %) and X-phos (19 mg, 20 mol %). Then dioxane (5 mL) was added under N₂. The Schlenk tube was screw capped and heated to 100° C. for 12 hours. Then the reaction mixture was cooled to room temperature, and sat. NH₄Cl aq. was poured into the reaction mixture and extracted with EtOAc. The organic layer was washed with brine, dried over Na₂SO₄, and filtered. The filtrate was concentrated in vacuo and the residue was purified by flash column chromatography to afford 15 (50 mg, 70%) as a white solid. ¹H NMR (400 MHZ, DMSO-d6) δ 9.35 (s, 1H), 8.40 (d, J=4.7 Hz, 2H), 7.57 (d, J=8.9 Hz, 2H), 6.90 (d, J=8.9 Hz, 2H), 6.74 (t, J=4.8 Hz, 1H), 3.45 (t, J=5.0 Hz, 4H), 3.00 (t, J=5.1 Hz, 4H), 1.42 (s, 9H). MS (EI): m/z 356.2 [M+H]+.

### 2-(2,6-Dioxopiperidin-3-yl)-4-((2-oxo-2-(4-(4-(pyrimidin-2-ylamino)phenyl)piperazin-1-yl)ethyl)amino) isoindoline-1,3-dione (PS-3)

In a 25 mL flask was added 15 (18 mg, 0.05 mmol) in TFA/DCM (5 mL, 1/1). The mixture was stirred for 30 min at room temperature. Then the solvent was removed in vacuo to give the deprotected intermediate, which was used for next step without further purification. To the above intermediate was added compound PS-6 (33 mg, 0.1 mol), HATU (38 mg, 0.1 mmol) and DIPEA (32 mg, 0.25 mmol) in DMF (2 mL). The mixture was stirred at room temperature for 30 min. Then the reaction mixture was concentrated in vacuo and the residue was purified by PrepHPLC with a reverse phase C18 column to afford the product as a yellow solid PS-3 (13 mg, 45%). ¹H NMR (400 MHZ, DMSO-d6) δ 11.11 (s, 1H), 9.37 (s, 1H), 8.41 (d, J=4.8 Hz, 2H), 7.65-7.52 (m, 3H), 7.15-7.03 (m, 3H), 6.94 (d, J=9.0 Hz, 2H), 6.75 (t, J=4.8 Hz, 1H), 5.07 (dd, J=12.9, 5.4 Hz, 1H), 4.26 (d, J=4.5 Hz, 2H), 3.67 (d, J=10.6 Hz, 4H), 3.10 (d, J=26.3 Hz, 4H), 2.95-2.80 (m, 1H), 2.67-2.55 (m, 2H), 2.09-1.97 (m, 1H). ¹³C NMR (100 MHZ, DMSO-d6) δ 173.3, 170.5, 169.3, 167.8, 166.9, 160.6, 158.4, 146.3, 145.9, 136.6, 133.7, 132.5, 120.7, 118.7, 117.1, 112.1, 111.3, 110.0, 49.9, 49.6, 49.0, 44.1, 41.9, 31.4, 22.6. HRMS (m/z): [M+H]+ calcd. for C₂₉H₂₉N₈O₅, 569.2261; found: 569.2249.

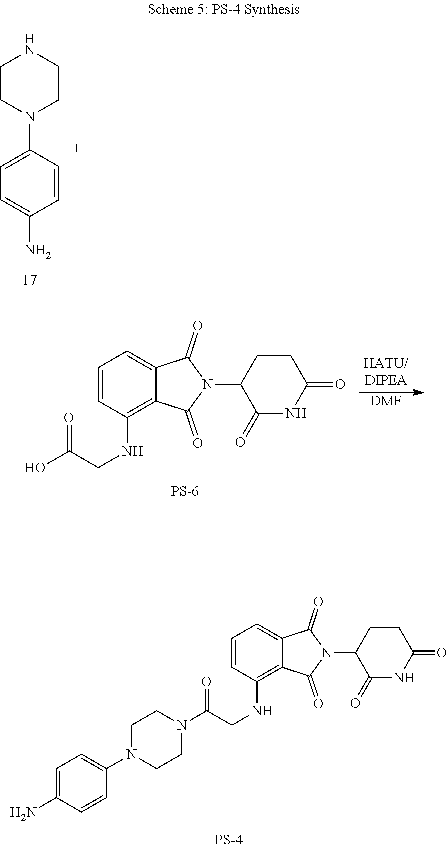

### 4-((2-(4-(4-Aminophenyl)piperazin-1-yl)-2-oxoethyl)amino)-2-(2,6-dioxopiperidin-3-yl) isoindoline-1,3-dione (PS-4)

To a 100 mL flask was added compound 16 (18 mg, 0.1 mmol), PS-6 (50 mg, 0.15 mmol), HATU (57 mg, 0.15 mmol) and DIPEA (65 mg, 0.5 mmol) in DMF (2 mL). The mixture was stirred at room temperature for 30 min. Then the reaction mixture was concentrated in vacuo and the residue was purified by PrepHPLC with a reverse phase C18 column to afford the product as a yellow solid PS-4 (24 mg, 50%). ¹H NMR (400 MHZ, DMSO-d6) δ 11.11 (s, 1H), 7.95 (s, 2H), 7.61 (t, J=7.8 Hz, 1H), 7.19-7.00 (m, 3H), 6.79-6.67 (m, 1.5H), 6.57-6.40 (m, 1.5H), 5.07 (dd, J=12.8, 5.4 Hz, 1H), 4.23 (d, J=4.6 Hz, 2H), 3.62 (dt, J=12.2, 4.9 Hz, 4H), 2.99-2.89 (m, 4H), 2.85 (d, J=5.7 Hz, 1H), 2.64-2.52 (m, 2H), 2.04 (m, 1H). ¹³C NMR (100 MHZ, DMSO-d6) δ 173.3, 170.5, 167.9, 166.8, 162.8, 145.9, 143.3, 142.4, 136.6, 132.5, 119.2, 118.7, 115.1, 111.3, 110.0, 51.3, 51.0, 49.0, 44.4, 44.1, 42.2, 31.2, 22.6. HRMS (m/z): [M+H]+ calcd. for C₂₅H₂₇N₆O₅, 491.2043; found: 491.2042.

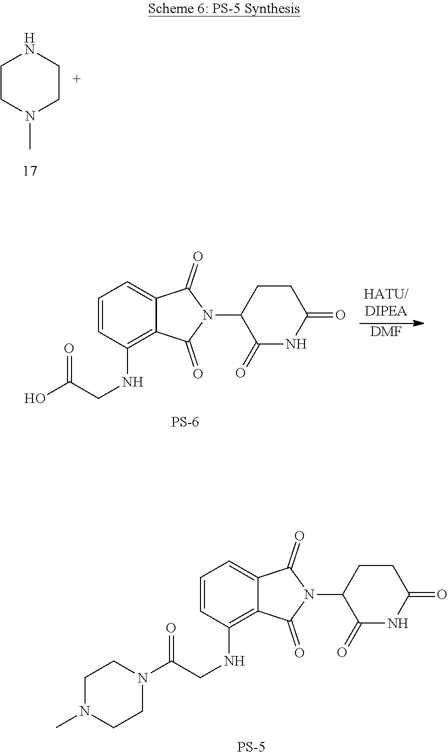

### 2-(2,6-Dioxopiperidin-3-yl)-4-((2-(4-methylpiperazin-1-yl)-2-oxoethyl)amino) isoindoline-1,3-dione (PS-5)

To a 100 mL flask was added compound 17 (10 mg, 0.1 mmol), PS-6 (50 mg, 0.15 mmol), HATU (57 mg, 0.15 mmol) and DIPEA (65 mg, 0.5 mmol) in DMF (2 mL). The mixture was stirred at room temperature for 30 min. Then the reaction mixture was concentrated in vacuo and the residue was purified by PrepHPLC with a reverse phase C18 column to afford the product as a yellow solid PS-5 (27 mg, 65%). ¹H NMR (400 MHZ, DMSO-d6) δ 11.11 (s, 1H), 7.60 (dd, J=8.5, 7.1 Hz, 1H), 7.12-7.04 (m, 3H), 5.07 (dd, J=12.9, 5.3 Hz, 1H), 4.18 (d, J=4.5 Hz, 2H), 3.49 (dt, J=10.2, 4.9 Hz, 4H), 2.89 (ddd, J=17.6, 13.9, 5.5 Hz, 1H), 2.64-2.51 (m, 2H), 2.33 (dt, J=25.4, 5.0 Hz, 4H), 2.21 (s, 3H), 2.07-1.99 (m, 1H). ¹³C NMR (100 MHZ, DMSO-d6) δ 173.3, 170.5, 169.2, 167.8, 166.8, 145.9, 136.6, 132.5, 118.7, 111.2, 110.0, 54.9, 54.6, 49.0, 46.0, 44.1, 44.0, 41.9, 31.4, 22.6 HRMS (m/z): [M+H]+ calcd. for C₂₀H₂₄N₅O₅, 414.1777; found: 414.1768.

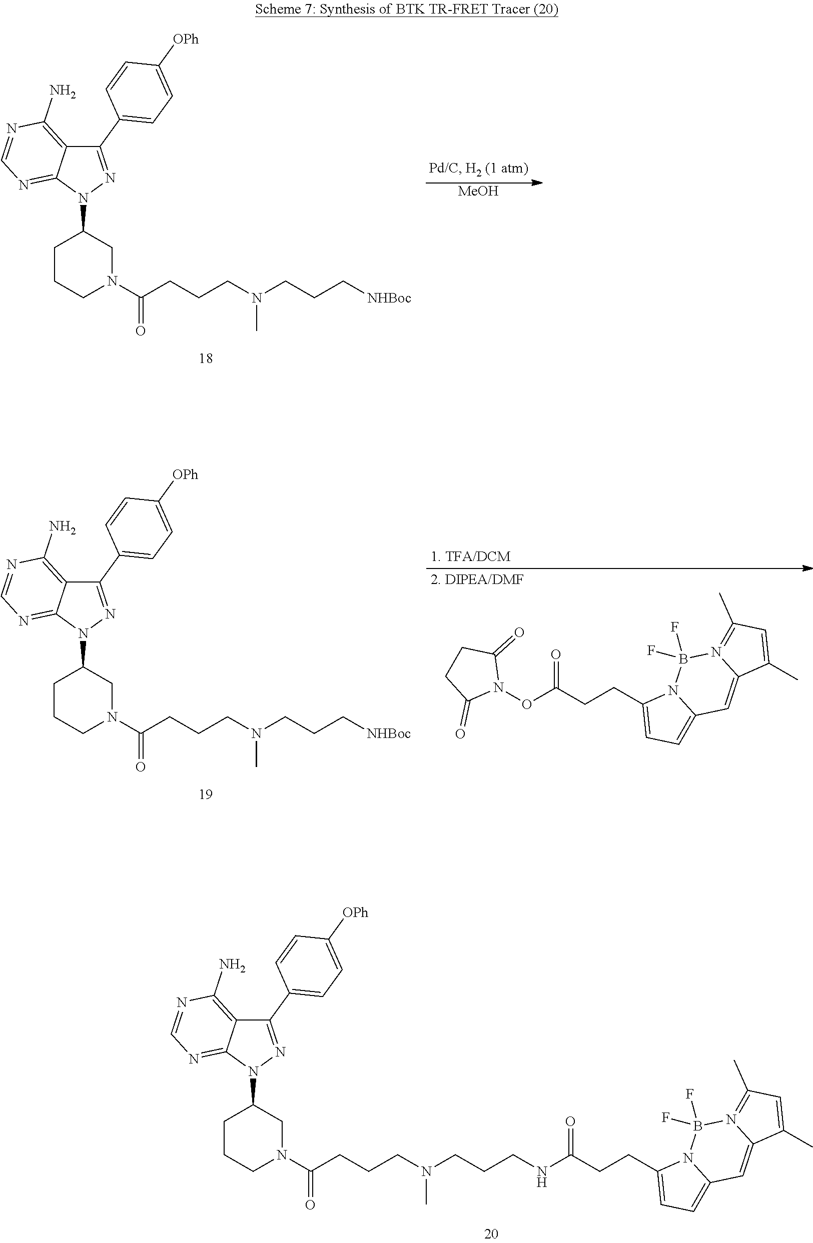

### tert-Butyl (R)-(3-((4-(3-(4-amino-3-(4-phenoxyphenyl)-1H-pyrazolo[3,4-d]pyrimidin-1-yl)piperidin-1-yl)-4-oxobutyl)(methyl)amino) propyl) carbamate (19)

To a flask was added compound 18 (6.5 mg, 0.01 mmol) and Pd/C (1 mg) in MeOH (2 mL). The mixture was stirred under 1 atm H₂ at room temperature overnight. LC-MS showed compound 18 converted into compound 19 completely. Then the reaction mixture was filtered, and the filtrate was concentrated in vacuo to provide compound 19 (5.5 mg, 90%) without further purification.

### BTK TR-FRET Tracer (20)

In a 25 mL flask was added 19 (5 mg, 0.05 mmol) in TFA/DCM (5 mL, 1/1). The mixture was stirred for 30 min at room temperature. Then the solvent was removed in vacuo to give the deprotected intermediate, which was used for next step without further purification. To the above intermediate was added BODIPY-FL (2 mg, 0.005 mol), and DIPEA (3.2 mg, 0.025 mmol) in DMF (2 mL). The mixture was stirred at room temperature for 30 min. Then the reaction mixture was concentrated in vacuo and the residue was purified by PrepHPLC with a reverse phase C18 column to afford the product as a dark blue solid 20 (2 mg, 50%). MS (EI): m/z 816.4.

### Example 2

### Materials and Methods

### Cell Line Engineering and Culture

HEK-293T17 cells were engineered to stably express nLuc-fused to the C-terminal of BTK or IKZF1 or IKZF3 via lentivectors. Briefly, BTK/IKZF1/IKZF3 expression constructs (DNASU: Cat. No. HsCD00514411, HsCD00512327, and HsCD00513042) were Gateway cloned into pLenti6.2-ccdB-Nanoluc vector (Addgene: Cat. No. 87078), which were co-transfected into HEK-293T cells with lentiviral envelope protein construct pCMV-VSV-G (Addgene, Cat. No. 8454) and lentiviral packaging plasmid (Addgene, Cat. No. 8454) for viral production. Transfections were carried out using calcium phosphate-mediated transfection method (Promega, Cat. No. E1200). Virus was harvested at 48 hours and 72 hours post transfection and transfection was performed in the presence of 8 μg/mL of polybrene. HEK-293T17 cells were treated with the concentrated viral particles. Following lentiviral transduction, stable cell lines were selected by 7.5 μg/mL of blasticidin. Successful establishment of stable cell lines were confirmed with Nano-Glo Luciferase Assay (Promega, Cat. No. N1110).

HEK-293T17, Mino, Jeko-1, Ramos, and A20 were obtained from the American Type Culture Collection (ATCC, Cat. No. CRL-11268, CRL-3000, CRL3006, CRL1596, and TIB-208). MOLM14 and Rec-R cells were obtained from non-commercial sources.

All cell lines were cultured in RPMI-1640 medium (Thermo Fisher Scientific, Cat. No. MT10040CV), except engineered HEK-293T17-BTK/IKZF1/IKZF3-nLuc cell lines, which were cultured in DMEM medium (Thermo Fisher Scientific, Cat. No. MT10013CV). All media contained 10% fetal bovine serum (GE Healthcare, Cat. No. SH30071.03) plus 1% Pen/Strep (Thermo Fisher Scientific, Cat. No. 15140163). All cells were grown in a humidified incubator at 37° C. with 5% CO₂.

### Immunoblotting

Mino cells were seeded into the wells of six-well plates at the density of 5×10⁵ cells per mL in 2 mL of complete RPMI-1640 culture medium. After overnight adaptation, cells were treated with serially diluted compounds (from 1,000-1.6 nM, 5-fold dilution) for 24 hours. After treatment, whole cell lysates for immunoblotting were prepared by pelleting Mino cells at 4° C. and 200×g for 5 minutes. The resulting cell pellets were washed once with ice-cold PBS and lysed in 1×RIPA lysis buffer (Alfa Aesar, Cat. No. J62524) supplemented with protease and phosphatase inhibitor cocktail (Thermo Fisher, Cat. No. 78430). Lysates were centrifuged at 15,000×g for 10 min at 4° C. and protein concentrations were assessed using BCA assay (Pierce, Cat. No. 23225). Same amounts of protein (30 μg) for each sample were loaded onto sodium dodecyl sulfate-polyacrylamide gel, separated by electrophoresis (Bio-Rad) at 120 V for 1.5 h and transferred to PVDF membrane using a Transblot Turbo system (Bio-Rad). After blocking for 1 h at room temperature in 1% BSA-TBST, the membranes were immunoblotted with the specified primary antibodies at the dilution of 1:1000 in TBST (Cell Signaling Technology: anti-BTK Cat. No. 8547, anti-IKZF1 Cat. No. 14859, anti-IKZF3 Cat. No. 15103, anti-β-actin Cat. No. 4570, Proteintech: anti-GSPT1, Cat. No. 10763-1-AP) overnight at 4° C. and the HRP-conjugated secondary antibodies (Cell Signaling, Cat. No. 7074, 1:1000 in TBST) for 1 h at room temperature. Imaging was performed using the ECL Prime chemiluminescent Western blot detection reagents (Kindle Biosciences, Cat. No. R1100) by visualization of the blots with an Imager (Kindle Biosciences, Cat. No. D1001). All Western blots were subsequently processed and quantified with Imager software ImageJ and protein level was normalized to the B-actin loading controls.

### Nanoluc-Based Endpoint and Protein Degradation Kinetics Assays

For endpoint assays, engineered HEK-293T17 cells were plated in white opaque 96-well plate (Thermo Fisher) at a density of 4,000 cells per well in either 100 μL of Opti-MEM (4 h treatment) or 100 μL of DMEM whole medium (24 h treatment). The nanoluc activity was determined using the furimazine substrate (Promega, Cat. No. N1110). Briefly, after 4 h incubation of indicated compounds, cells were directly lysed by addition of 100 μL of nanoluciferase detection solution, containing lysis buffer and furimazine (1/50 dilution). For the 24-hour treatment group, the old medium was replaced with 100 μL fresh opti-MEM, followed by the addition of equal volume of lysis buffer plus furimazine (1/50 dilution). The endpoint luminescence was measured with a microplate reader (BioTek Synergy H1).

For protein degradation kinetics assays, HEK-293T17 cells stably expressing nLuc fusion proteins were plated in white opaque 96-well plates (Thermo Fisher) at a density of 4,000 cells per well in 100 μL of Opti-MEM. Cells were allowed to attach overnight and treated with 40 nM or 200 nM of indicated compounds plus endurazine (1:2000, Promega, Cat. No. N2570) and extracellular nanoluc inhibitor (1:2000, Promega, Cat. No. N2162), after which the real-time luminescence was measured with a microplate reader (BioTek Synergy H1) immediately every 5 min for 24 hours. The kinetics data were fitted in the “one phase decay” model in Prism 9 to calculate the half-life, defined as time to reach 50% of maximal protein degradation.

### Cell Viability Assays

Mino cells were plated in 96-well plates (Corning, Cat. No. 3598) at a density of 6,000 cells per well in 100 μL of medium. Cells were treated the next day with indicated compounds in a five-fold dilution series (from 1000 to 0.64 nM), followed by 72 h incubation at 37° C. with 5% CO₂. Cell viabilities were measured using the Alarma Blue assay by adding pre-warmed Resazuerin sodium (Sigma, Cat. No. 199303) solution (1 mg mL⁻¹ in PBS) in an amount equal to 10% of the volume in the well. Four hours after incubation, fluorescence signals were measured with a BioTek Synergy H1 microplate reader at excitation/emission 544/590 nm from top with a gain of 60. The EC₅₀ values were calculated using GraphPad Prism 9.3 (GraphPad Software, La Jolla, CA) with the nonlinear fitting model of “[Inhibitor] vs. response—Variable slope (four parameters)”. The IC₅₀ values, defined as compound concentrations that reduce cell viabilities by 50%, were extrapolated based on the nonlinear fitting model and reported in figure panels.

### TR-FRET Biochemical Binding Assays

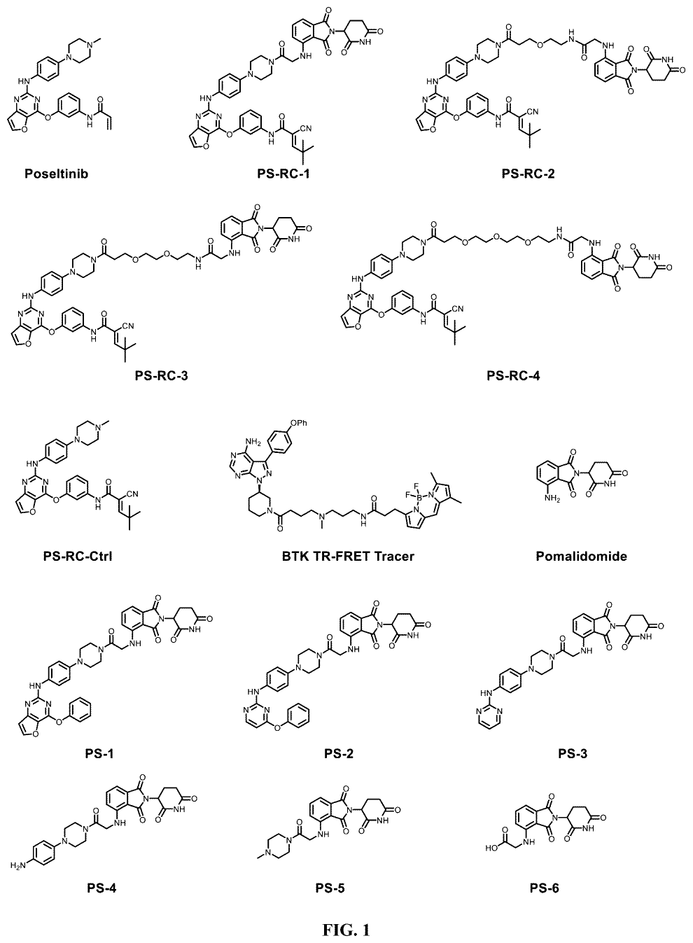

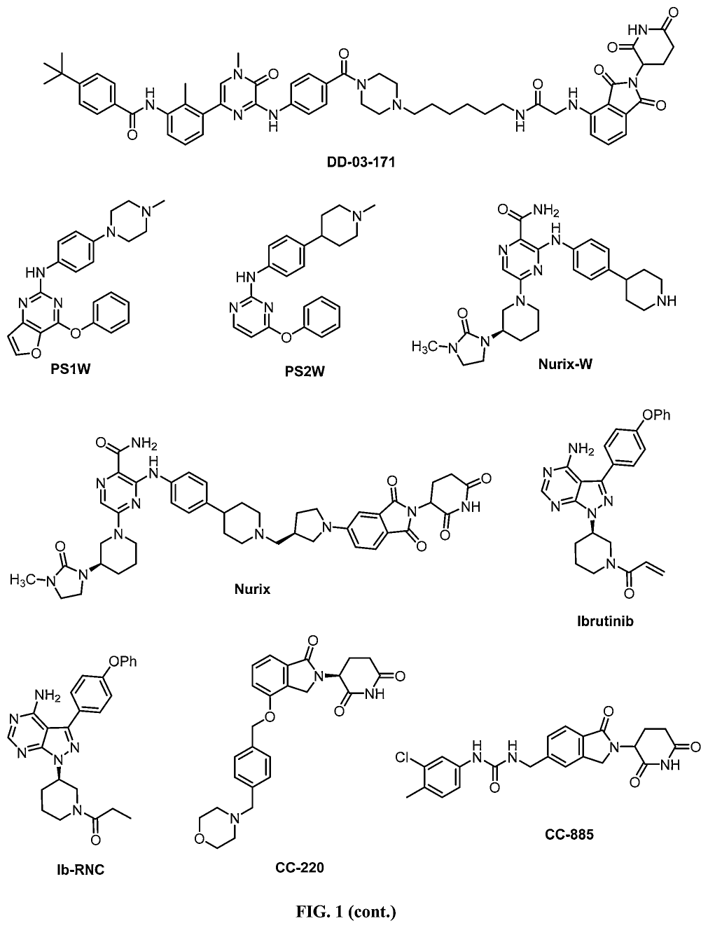

Time-resolved fluorescence resonance energy transfer (TR-FRET) assay was carried out to evaluate the binding of indicated compounds and BTK by competition with a BODIPY-FL labeled BTK tracer (See FIG. 1, BTK TR-FRET tracer). The assay was performed in 20 μL assay buffer (50 mM Tris, pH7.5, 0.1% Triton X-100, 0.01% BSA, and 1 mM DTT) with 0.3 nM Tb-anti-His (Cisbio, Cat. No. 61HI2TLA), 2 nM His-BTK (SignalChem, Cat. No. B10-10H-10), 150 nM BTK-BODIPY tracer and serial diluted compounds (10,000-0.64 nM, 5-fold dilutions) in opaque 384-well plates. Unless specified otherwise, all assays were performed in triplicates. The assay mixtures were incubated at room temperature in the dark for 120 min, unless specified otherwise, and the signals were collected using a BioTek Synergy H1 microplate reader to measure the fluorescence emission ratio (1520 nm/1490 nm) of each well, using a 340-nm excitation filter, a 100-μs delay, and a 200-μs integration time. Raw data from the plate reader were used directly for analysis. The curve-fitting software GraphPad Prism 9 was used to generate graphs and curves and to determine IC₅₀ values.

### Proteomics

Two million of Mino cells were treated with DMSO or 200 nM of PS-2 for 4 hours in biological triplicates and cells were harvested by centrifugation (200×g, 4° C., 5 min). Lysis buffer (100 mM Triethylammonium bicarbonate (TEAB, Thermo Fisher Scientific, Cat. No. 90114), 2% SDS) was added to the cell pellets and homogenized with a microtip sonicator (Branson) to achieve a cell lysate with a protein concentration of ˜2 mg mL⁻¹. Protein concentration was determined using BCA assay (Pierce, Cat. No. 23225) and normalized to 1 mg mL⁻¹. One hundred μg of protein for each sample were reduced and alkylated with 10 mM Tris(2-carboxyethyl) phosphine hydrochloride (TCEP, Sigma, Cat. No. C4706) and 17 mM iodoacetamide (IAA, Sigma, Cat. No. 16125-5G), followed by digestion with trypsin (1:20, enzyme: protein, Thermo Fisher Scientific, Cat. No. 90058) for 16 h shaking at 37° C. Tandem mass tag (TMT) reagents (Thermo Fisher Scientific, Cat. No. A44520) were dissolved in anhydrous acetonitrile (ACN, Sigma, Cat. No. 271004), 0.08 mg of label reagent was used for 10 μg of protein digest. The 16-plex labeling reactions were performed for 1 hour at room temperature and the reaction was quenched by the addition of 5% hydroxylamine solution for 15 min at room temperature. The sample channels were combined at a 1:1:1:1:1:1 ratio, desalted using peptide desalting columns (Thermo Fisher Cat. No. 89852). After drying down in a speed vacuum overnight, the combined sample was resuspended in 1% formic acid and subjected to Orbitrap Fusion Lumos mass spectrometer (Thermo Fisher Scientific, San Jose, CA, USA).

### Results

### Development of Reversible Covalent BTK PROTACs

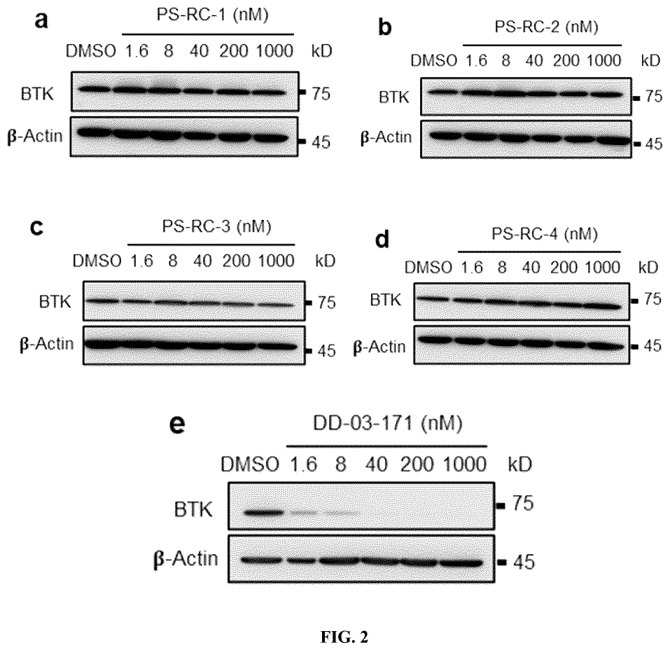

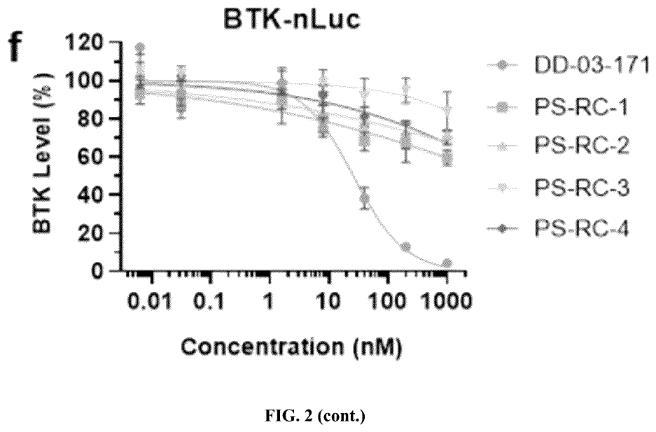

A series of reversible covalent BTK PROTACs with different linkers were synthesized (FIG. 1). PS-RC-1 has a rigid linker, while PS-RC-2, PS-RC-3 and PS-RC-4 share flexible PEG linkers with different lengths. To test whether the four PROTACs can induce BTK degradation, Mino cells were treated with PS-RC-1, PS-RC-2, PS-RC-3 and PS-RC-4 for 24 hours, followed by western blotting to quantify the total BTK levels. Surprisingly, none of the tested PROTACs induce appreciable BTK degradation, while the positive known control compound DD-03-171 (Dobrovolsky et al., Blood, 133:952-961 (2019); see FIG. 1) potently induces BTK degradation (see FIG. 2a-e, Table 1). Poseltinib (FIG. 1) was also used as a control. To corroborate with the Western blot results, the compounds were tested in HEK-293 cells stably expressing a BTK-Nano-Luciferase (nLuc) fusion protein as a reporter and similar results were found (FIG. 2f, Table 1). The DC₅₀ (concentration of PROTACs required to achieve 50% degradation of the target protein) and Dₘₐₓ (maximum level of target protein can be degraded by PROTACs) values obtained through this assay are listed in Table 1.

TABLE 1
Degradation of BTK induced by reversible
covalent BTK PROTACs.

| | | | | | BTK |
|---|---|---|---|---|---|
| | BTK-nLuc | | BTK WB | | binding |
| | DC₅₀ | Dₘₐₓ | DC₅₀ | Dₘₐₓ | IC₅₀ |
| Compounds | (nM) | (%) | (nM) | (%) | (nM) |
| DD-03-171 | 25.9 | 96 | 0.9 | 99 | — |
| PS-RC-1 | >1,000 | 40 | >1,000 | — | >10,000 |
| PS-RC-2 | >1,000 | 30 | >1,000 | — | >10,000 |
| PS-RC-3 | >1,000 | 16 | >1,000 | — | 4,367 |
| PS-RC-4 | >1,000 | 30 | >1,000 | — | 6,614 |
| Poseltinib | — | — | — | — | 1.21 |
| PS-RC-Ctrl | — | — | — | — | 869 |

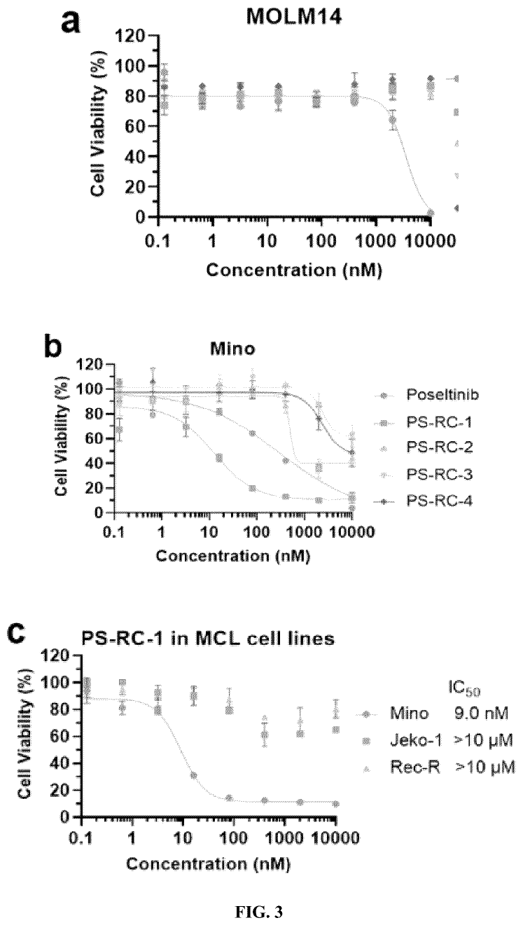

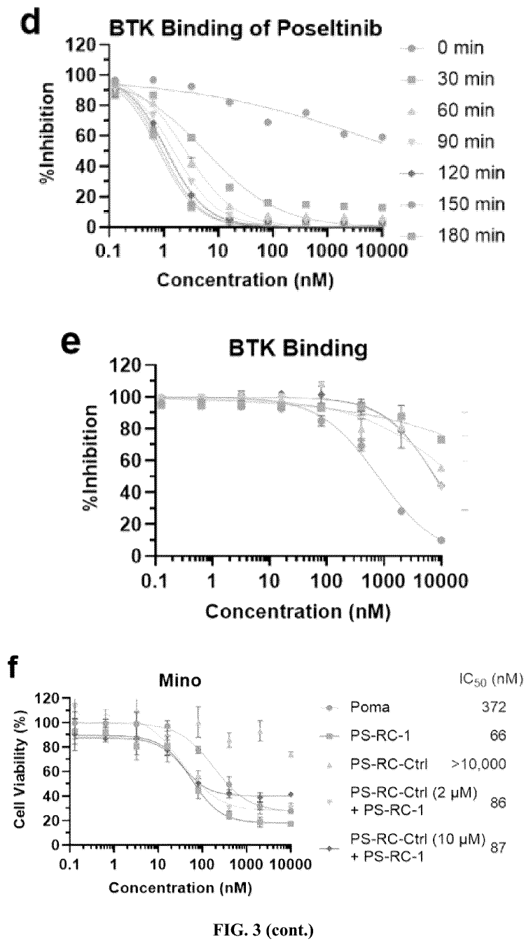

The potencies of PS-RC-1, PS-RC-2, PS-RC-3 and PS-RC-4 in inhibiting cell growth were assessed with poseltinib as a positive control (FIG. 3a-b). In MOLM-14 cells, an acute monocytic leukemia (AML) cell line, the IC₅₀ value of poseltinib was 2.9 μM, while all the reversible covalent BTK PROTACs did not show any appreciable inhibitory effects in the same cells at 10 μM, the highest concentration tested (FIG. 3a). In Mino cells, a mantle cell lymphoma (MCL) cell line, PS-RC-1 is highly potent with an IC₅₀ value of 12 nM. In contrast, PS-RC-2, PS-RC-3 and PS-RC-4 barely inhibit 50% of cell growth at 10 μM in Mino cells (FIG. 3b). To test whether the potent inhibitory effect of PS-RC-1 is MCL cell specific, its potency was tested in two additional MCL cell lines, Jeko-1 and Rec-R, but it was found that these two MCL cells poorly respond to PS-RC-1 (FIG. 3c). Based on these data and not to be bound by theory, the growth inhibitory effect of PS-RC-1 in Mino cells may not be due to BTK degradation but related to a specific cell signaling pathway in this cell line.

### Biochemical Binding Affinities of Reversible Covalent PROTACs to BTK

To measure the binding affinities between PROTACs and BTK, a time-resolved fluorescence resonance energy transfer (TR-FRET) competition binding assay was developed. A BTK tracer was synthesized by conjugating an ibrutinib-based reversible noncovalent binder with the BODIPY-FL dye (FIG. 1). In a saturation binding assay, it was shown that the BTK tracer has a K_{d} value of 155 nM to the recombinant BTK protein. In the presence of the BTK tracer, competition assays were performed to measure the binding affinities between BTK and its inhibitors and PROTACs. As an irreversible covalent inhibitor, poseltinib is highly potent with an IC₅₀ value of 1.1 nM for 2 hours of incubation (FIG. 3d). Surprisingly, substituting the acrylamide in poseltinib with a reversible covalent cyano-acrylamide group (referred to herein as “PS-RC-Ctrl”) dramatically decreases the binding affinity to BTK by >700 folds (IC₅₀ 1.2 nM vs 869 nM, Table 1). Converting the reversible covalent cyano-acrylamide containing poseltinib to PROTACs further decreases their binding affinities to BTK (IC₅₀>7.5 μM, FIG. 3e, Table 1). The very weak BTK binding affinities for PS-RC-1, PS-RC-2, PS-RC-3 and PS-RC-4 explain their minimal BTK degradation effects in cells and further support that the observed growth inhibitory effect of PS-RC-1 in Mino cells is unrelated to the BTK pathway.

### Examining Off-Target Effects of CRBN Engaged PROTAC's

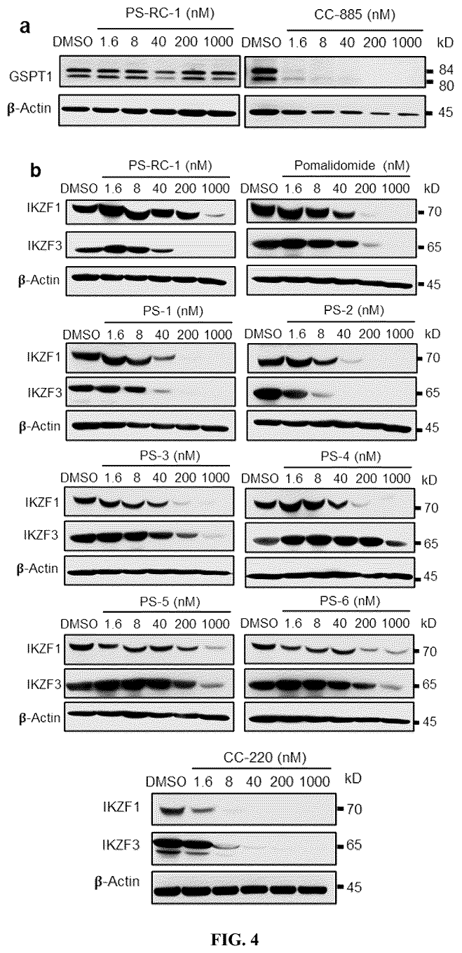

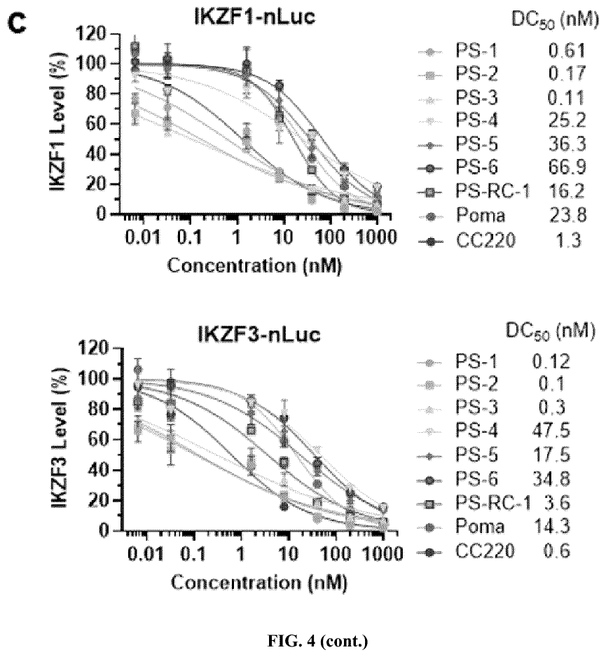

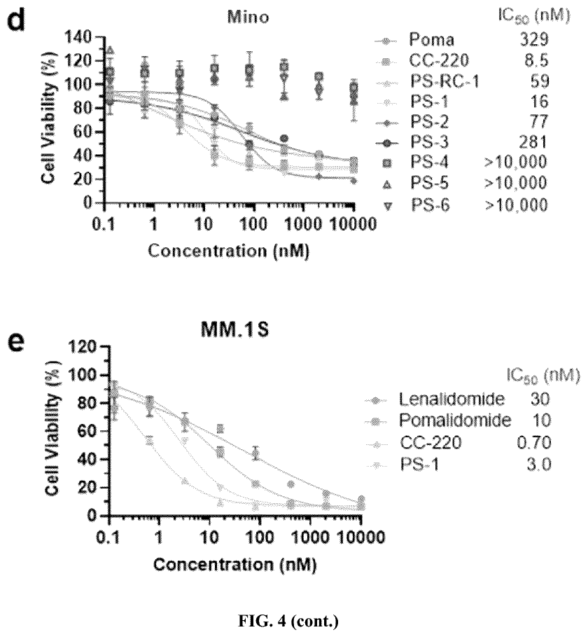

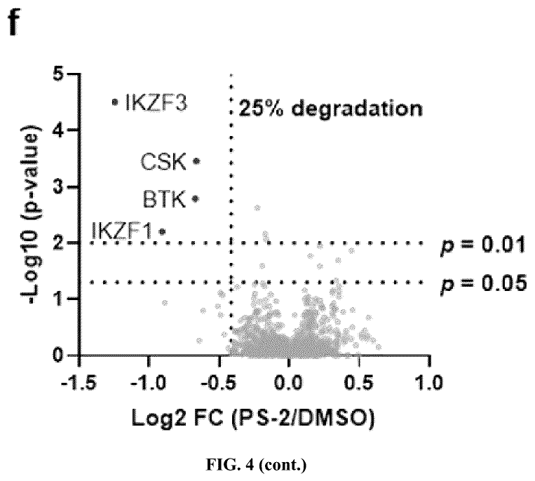

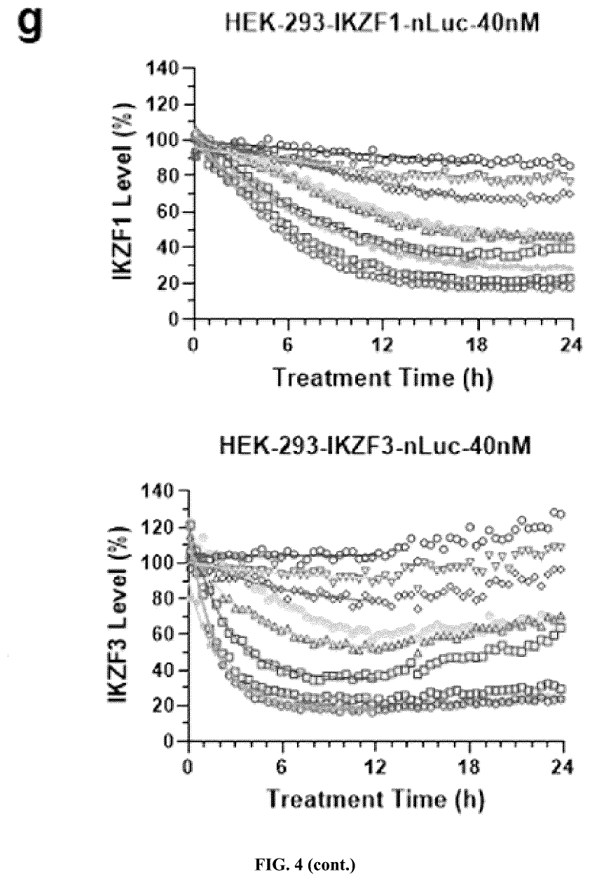

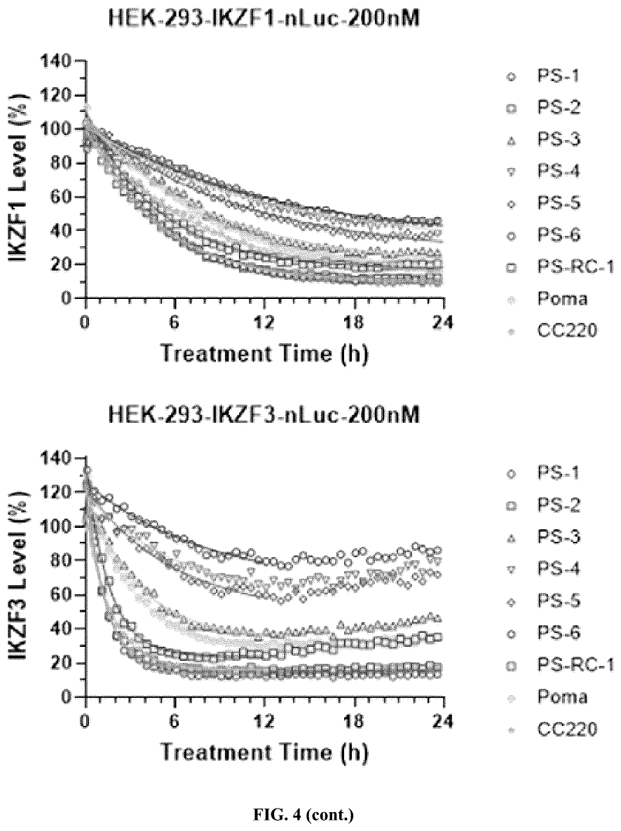

CC-885 and CC-220, two commercially available derivatives of lenalidomide, potently degrade the cell cycle regulator and translation termination factor GSPT1 as a neo-substrate for Cereblon. GSPT1 is known as a common off-target for phthalimide-based PROTACs. Additionally, depending on the linker position on thalidomide, the CRBN engaged PROTACs can also degrade IKZF1 and IKZF3. To test these three off-target degradations, Mino cells were treated with PS-RC-1 at different concentrations for 24 hours, followed by Western blotting. PS-RC-1 does not degrade GSPT1 (FIG. 4a) but potently degrades IKZF3 (IC₅₀=44 nM, FIG. 4b and Tables 2A and 2B) and IKZF1 to a lesser extent (IC₅₀=802 nM, FIG. 4b and Tables 2A and 2B). Immunomodulatory drugs (IMiDs), such as pomalidomide and lenalidomide, have been explored to treat relapsed or refractory mantle-cell lymphoma (Wang et al., 2012), presumably due to its degradation effect of IKZF1/3. Mino, Jeko-1 and Rec-R cells were treated with pomalidomide and it was found that only Mino cells are responsive to pomalidomide (IC₅₀=329 nM, FIG. 3d), which is less potent than PS-RC-1 (IC₅₀=9.0 nM, FIG. 3c) in the same cell line. Comparing the IKZF3 degradation efficiency, PS-RC-1 is more potent than pomalidomide (IC₅₀ 3.6 vs 14.3 nM (nLuc assay), DC₅₀ 44 vs 54 nM (Western blot), Tables 2A and 2B).

TABLE 2A
Degradation of IKZF1 induced by PS-RC-1 and its analogs.

| | | | IKZF1-nLuc Degradation Kinetics | | | | | |
|---|---|---|---|---|---|---|---|---|
| | IKZF1-nLuc | | 40 nM | | 200 nM | | IKZF1-WB | |
| | Endpoint | | Compounds | | Compounds | | Endpoint | |
| | DC₅₀ | Dₘₐₓ | | Dₘₐₓ | | Dₘₐₓ | DC₅₀ | Dₘₐₓ |
| Compounds | (nM) | (%) | t_{1/2} (h) | (%) | t_{1/2} (h) | (%) | (nM) | (%) |
| PS-RC-1 | 16 | 95 | 5.4 | 60 | 3.4 | 80 | 802 | 62 |
| Pomalidomide | 24 | 90 | 15 | 53 | 5.5 | 80 | 42 | 96 |
| CC220 | 1.3 | 92 | 7.2 | 72 | 5.1 | 82 | 3.1 | 95 |
| PS-1 | 0.6 | 98 | 4.3 | 83 | 3.4 | 90 | 44 | 99 |
| PS-2 | 0.2 | 97 | 4.9 | 80 | 3.4 | 89 | 28 | 99 |
| PS-3 | 0.1 | 94 | 15 | 52 | 5.6 | 75 | 91 | 90 |
| PS-4 | 25 | 82 | 23 | 20 | 8.3 | 56 | 146 | 86 |
| PS-5 | 36 | 85 | >24 | 33 | 7.2 | 63 | 464 | 64 |
| PS-6 | 67 | 80 | >24 | 12 | 8.4 | 50 | 384 | 65 |

TABLE 2B
Degradation of IKZF3 induced by PS-RC-1 and its analogs.

| | | | IKZF3-nLuc Degradation Kinetics | | | | | |
|---|---|---|---|---|---|---|---|---|
| | IKZF3-nLuc | | 40 nM | | 200 nM | | IKZF3-WB | |
| | Endpoint | | Compounds | | Compounds | | Endpoint | |
| | DC₅₀ | Dₘₐₓ | t_{1/2} | Dₘₐₓ | t_{1/2} | Dₘₐₓ | DC₅₀ | Dₘₐₓ |
| Compounds | (nM) | (%) | (h) | (%) | (h) | (%) | (nM) | (%) |
| PS-RC-1 | 3.6 | 91 | 1.6 | 43 | 1.0 | 65 | 44 | 92 |
| Pomalidomide | 14 | 94 | 5.1 | 52 | 1.9 | 64 | 54 | 96 |
| CC220 | 0.6 | 96 | 1.5 | 80 | 1.2 | 84 | 3.8 | 98 |
| PS-1 | 0.1 | 98 | 1.2 | 79 | 0.9 | 88 | 39 | 95 |
| PS-2 | 0.1 | 98 | 1.2 | 75 | 0.9 | 84 | 2.5 | 99 |
| PS-3 | 0.3 | 94 | 2.8 | 45 | 1.9 | 55 | 250 | 78 |
| PS-4 | 48 | 85 | >24 | 12 | >24 | 20 | >1,000 | 15 |
| PS-5 | 18 | 87 | >24 | 23 | >24 | 27 | >1,000 | 45 |
| PS-6 | 35 | 85 | >24 | — | >24 | 12 | 301 | 70 |

Not to be bound by theory, it is possible that the reversible covalent analog of poseltinib (PS-RC-Ctrl) may bind other off-targets, contributing to the observed cell growth inhibition effect of PS-RC-1 in Mino cells. It was found that PS-RC-Ctrl has no appreciable effect on cell growth in Mino cells. To test whether the phenotype of PS-RC-1 in Mino cells is due to its bivalent structure-based protein degradation or a monovalent molecular glue, Mino cells were pretreated with a large excess of PS-RC-Ctrl (2 and 10 μM) for 2 h to prevent ternary complex formation with PS-RC-1, followed by PS-RC-1 treatment for 24 h. An appreciable IC₅₀ shift was not observed for PS-RC-1 in Mino cells upon PS-RC-Ctrl pre-treatment, suggesting that the reversible covalent poseltinib moiety in PS-RC-1 does not significantly contribute to the observed toxicity in Mino cells (FIG. 3f). Therefore, PS-RC-1 is a potent IKZF1/3 molecular glue degrader.

### Optimization of IKZF1/3 Degrader

As PS-RC-1 degrades IKZF1/3 instead of BTK, it was determined whether the molecular size could be reduced by removing the BTK binding moiety. A series of PS-RC-1 analogs, PS-1, PS-2, PS-3, PS-4, PS-5 and PS-6 (FIG. 1), were developed by maintaining the pomalidomide group while systemically shrinking the reversible covalent poseltinib moiety to explore the structure-activity relationship. The IKZF1 and IKZF3 degradation potencies were tested for these compounds in both HEK-293 cells stably expressing nLuc labeled IKZF1 and IKZF3 and in Mino cells using Western blotting. PS-RC-1, PS-1, and PS-2 have similar potencies to induce IKZF1 and IKZF3 degradation. PS-3, a PS-2 analog without the phenoxyl group, PS-4, PS-5 and PS-6 show significant reduction in the degradation potencies and preferential degradation of IKZF1 over IKZF3, similar to pomalidomide. Based on the Western blot in Mino cells, it was found that among these analogs, PS-2 is the most potent to degrade IKZF1 and IKZF3 (DC so 27.8 nM and 2.5 nM), but less potent than CC-220 (DC₅₀ 3.1 nM and 3.8 nM, FIG. 4b and Tables 2A and 2B), an IMiD developed by Celgene. PS-RC-1, PS-1, and PS-2 inhibits Mino cell growth with IC₅₀ values of 59, 16 and 77 nM, respectively (FIG. 4d).

IMiDs are commonly used to treat multiple myeloma (MM). PS-1 was further tested in MM.1S cells, an MM cell line. As shown in FIG. 4e, PS-1 is more potent than pomalidomide and lenalidomide in inhibiting the growth of MM.1S cells (IC₅₀=3.0 nM).

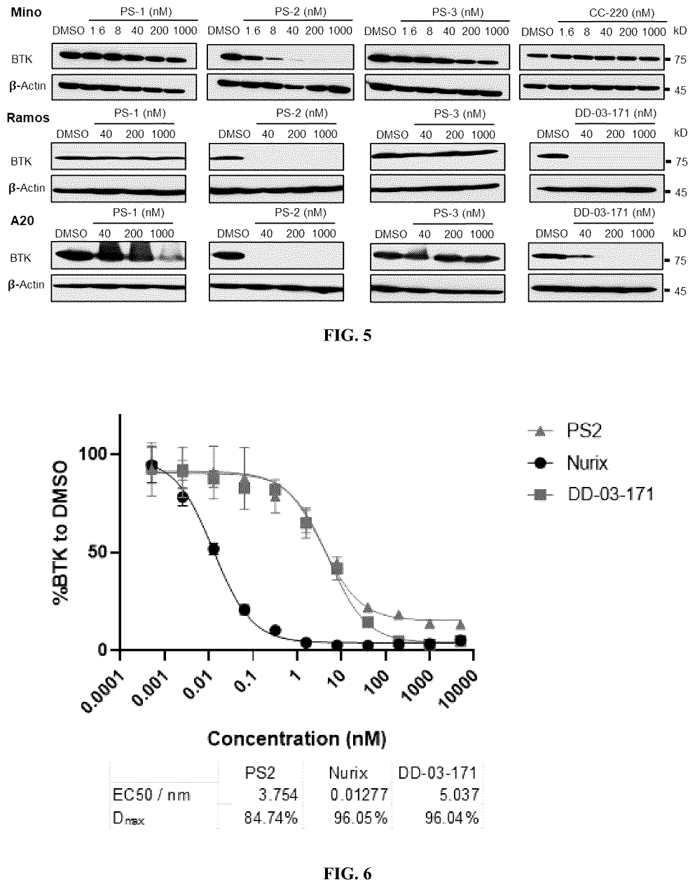

Next, tandem mass tagging (TMT)-based quantitative proteomic profiling was performed to evaluate the degradation specificity of PS-2. PS-2 was chosen for the proteomics study because it is the most potent to induce degradation of IKZF1 and IKZF3 in Mino cells. To avoid the potential secondary effects induced by PS-2 treatment, Mino cells were treated with 200 nM of PS-2 for 4 h. Quantitative proteomics employing TMT chemical labeling coupled with LC/MS/MS enabled the detection of >2,500 unique proteins without fractionation (FIG. 4f). Consistent with Western blots (FIG. 4b), PS-2 induces >60% degradation of IKZF3 and ˜50% degradation of IKZF1 after 4-h treatment, indicating the selectivity of PS-2 as a IKZF1/IKZF3 degrader. Two additional downregulated targets, BTK and CSK, were observed suggesting that downsized poseltinib can still bind to these two kinases. It was further validated that PS-2, but not PS-1 or PS-3, potently induces BTK degradation in Mino cells using Western blotting (FIG. 5), corroborating the proteomics data. No other proteins are downregulated by PS-2 for at least 25% with statistical significance, indicating that PS-2 degrades IKZF1/3 with high specificity in Mino cells.

Compared with PS-1, the BTK binding moiety of PS-2 lacks the furan ring fused to pyrimidine, which may be flexible enough to allow formation of a more stable ternary complex with BTK and CRBN in cells. In contrast, PS-3, whose warhead is further reduced compared with PS-2, loses its ability to function as either a BTK degrader or a potent IKZF1/3 molecular glue. To confirm this observation, BTK degradation induced by PS-1, PS-2, and PS-3 was tested in both a human B lymphocyte cell line Ramos and mouse BALB/c B cell lymphoma line A20, with DD-03-171 from the Gray group as a positive control (FIG. 5). Consistent with the results observed in Mino cells, only PS-2, but not PS-1 or PS-3, can induce potent BTK degradation in Ramos and A20 cells with a potency comparable or better than DD-03-171.

The degradation kinetics of IKZF1 and IKZF3 were further examined using the HEK-293 cells stably expressing IKZF1-nLuc or IKZF3-nLuc. PS-1 and PS-2 induce the fastest degradation of IKZF1 and IKZF3 with degradation half-lives of ˜2.8 and ˜0.8 h, respectively (FIG. 4g and Tables 2A and 2B), which is faster than pomalidomide (4.9 h and 1.9 h for IKZF1 and IKZF3, respectively) and generally consistent with the potency trends measured by endpoint nLuc assays and Western blotting.

To further illustrate the BTK degradation ability of PS-2, BTK-Hibit knock-in Ramos cells were treated with PS-2 for 24 hours, using Nurix and DD-03171 as controls (FIG. 6). The results showed that PS-2 has very potent BTK degradation abilities with a DC₅₀ of 3.754 nM and a Dₘₐₓ of 84.74%.

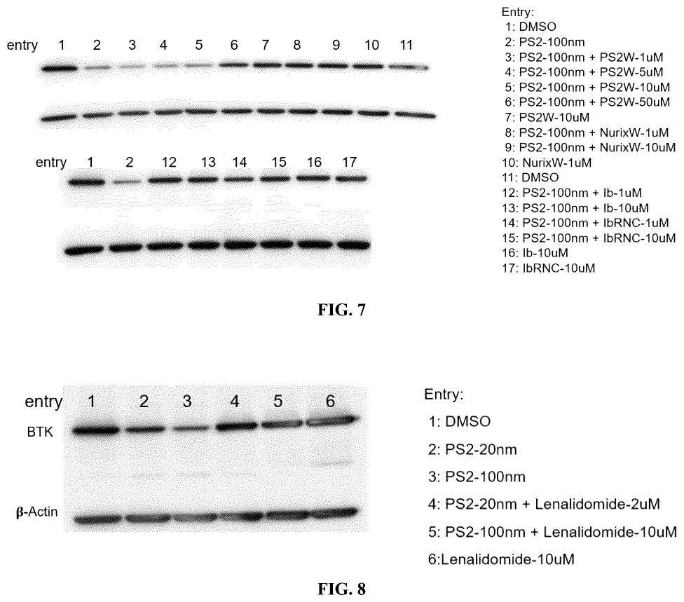

A BTK ligand competition assay was also conducted to shed light on the mechanism of PS-2. Ramos cells were incubated overnight, and then the indicated compounds were added and incubated for 24 hours, followed by Western blotting for BTK degradation (FIG. 7). Most BTK protein was degraded by 100 nM PS-2 (entry 2), but when potent BTK inhibitors such as Ibrutinib were added with PS-2, PS-2's BTK degradation ability was completely outcompeted (entry 12). Not to be bound by theory, this result indicates that PS-2 also binds to BTK to start the degradation process, with the binding site located in the same pocket as traditional inhibitors. The warhead of PS-2, which is the BTK-binding part of PS-2 and hereinafter referred to as PS2W, was also synthesized. It was found that even at a concentration of 50 μM (500 fold), PS2W cannot completely compete out the degradation potency of PS-2 (entry 6), indicating that PS-2's binding affinity to BTK is very weak. Competitive experiments were conducted with CRBN ligands, and the results showed that lenalidomide can compete with PS-2 for BTK degradation, indicating that PS-2 exerts its degradation ability by binding to CRBN (FIG. 8).

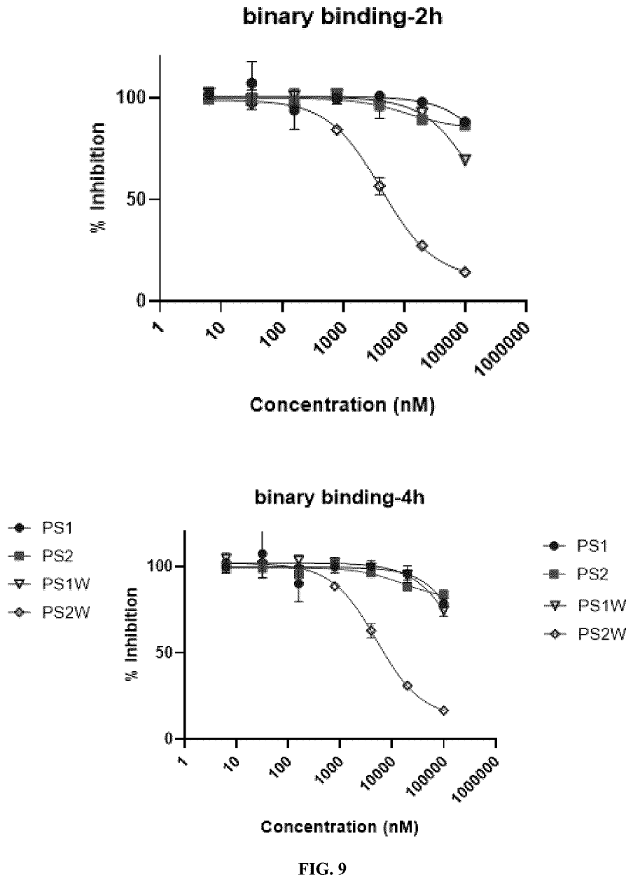

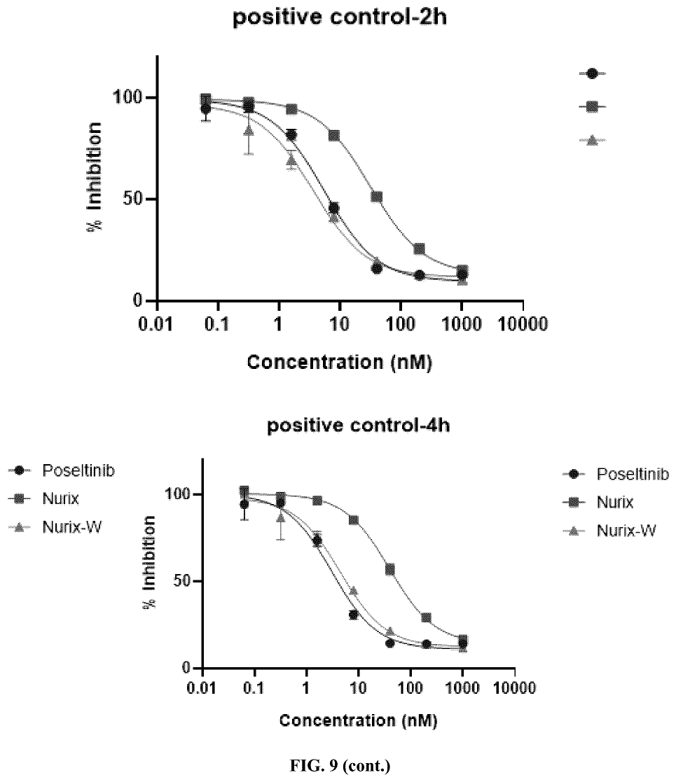

To quantify the binding ability of PS-2 to BTK, a TRFRET assay for BTK was performed (FIG. 9). The results showed that even at 100 μM, PS-1 and PS-2 showed almost no binding to BTK, while the positive control group Nurix and the inhibitor Poselbtinib showed binding affinity in the nM range.

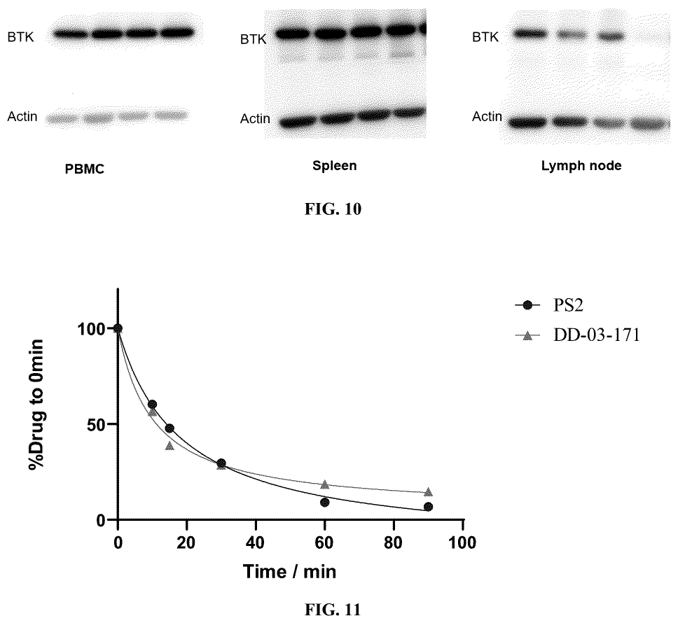

PD data results are shown in FIG. 10, where four mice were intraperitoneally administered PS-2 at a dose of 30 mg/kg once a day for three days. Their peripheral blood mononuclear cells (PBMC), spleen, and lymph nodes were collected and BTK levels were tested with Western Blot.

To verify the metabolic stability of PS-2, PS-2 was co-incubated with mouse liver S9 enzymes and LC-MS was used to test the remaining amount of PS-2 compound. DD-03-171 was used as a control (FIG. 11). The results showed that PS-2 is unstable with the presence of mouse S9 enzyme, with a t_{1/2} of only around 20 minutes.

The compounds and methods of the appended claims are not limited in scope by the specific compounds and methods described herein, which are intended as illustrations of a few aspects of the claims and any compounds and methods that are functionally equivalent are within the scope of this disclosure. Various modifications of the compounds and methods in addition to those shown and described herein are intended to fall within the scope of the appended claims. Further, while only certain representative compounds, methods, and aspects of these compounds and methods are specifically described, other compounds and methods are intended to fall within the scope of the appended claims. Thus, a combination of steps, elements, components, or constituents can be explicitly mentioned herein; however, all other combinations of steps, elements, components, and constituents are included, even though not explicitly stated.

What is claimed is:

1. A compound of the following formula: 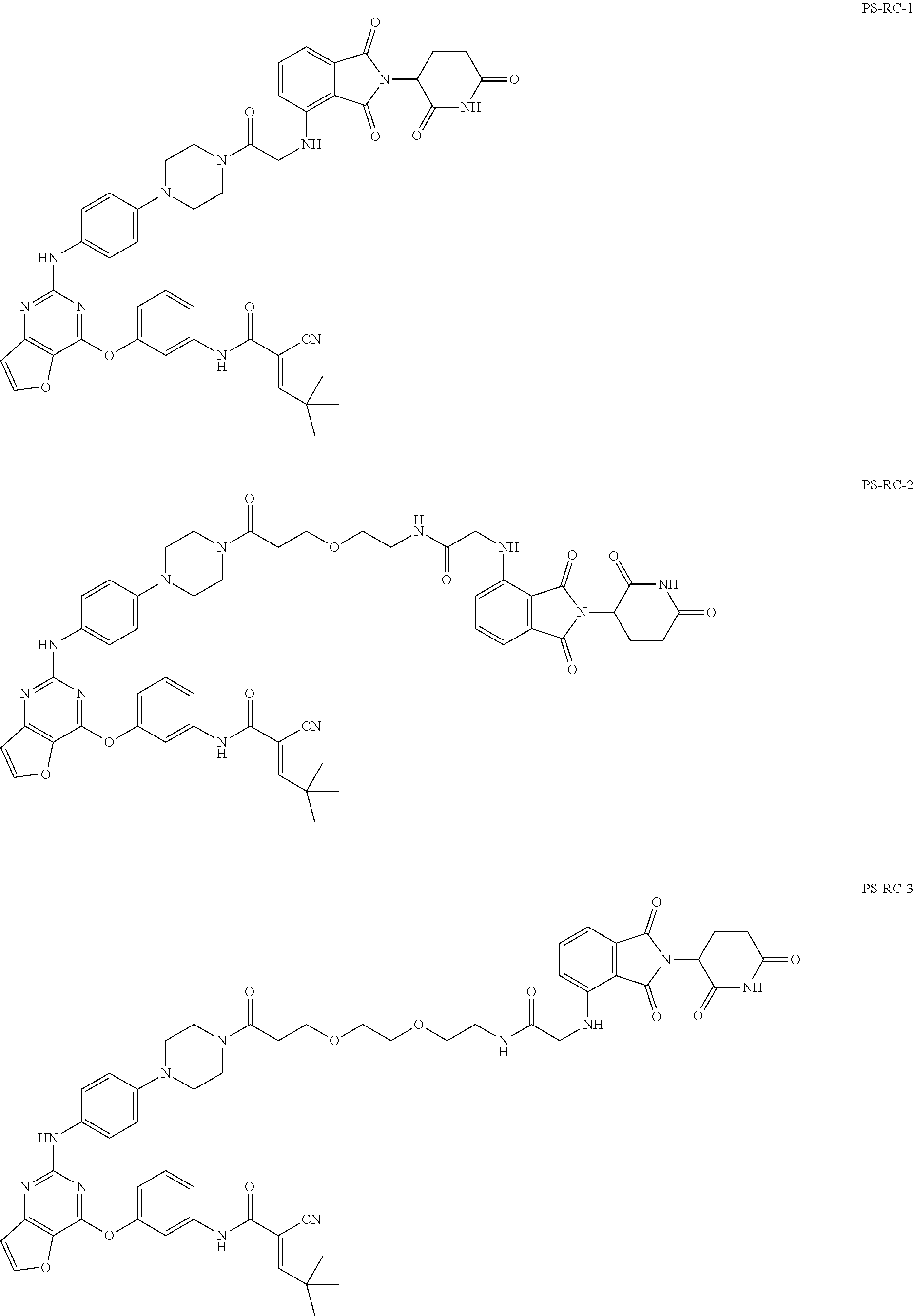 [SMILES: CC(C)(C)/C=C(\C#N)C(=O)Nc1cccc(Oc2nc(Nc3ccc(N4CCN(C(=O)CNc5cccc6c5C(=O)N(C5CCC(=O)NC5=O)C6=O)CC4)cc3)nc3ccoc23)c1 ; CC(C)(C)/C=C(\C#N)C(=O)Nc1cccc(Oc2nc(Nc3ccc(N4CCN(C(=O)CCOCCNC(=O)CNc5cccc6c5C(=O)N(C5CCC(=O)NC5=O)C6=O)CC4)cc3)nc3ccoc23)c1 ; CC(C)(C)/C=C(\C#N)C(=O)Nc1cccc(Oc2nc(Nc3ccc(N4CCN(C(=O)CCOCCOCCNC(=O)CNc5cccc6c5C(=O)N(C5CCC(=O)NC5=O)C6=O)CC4)cc3)nc3ccoc23)c1] 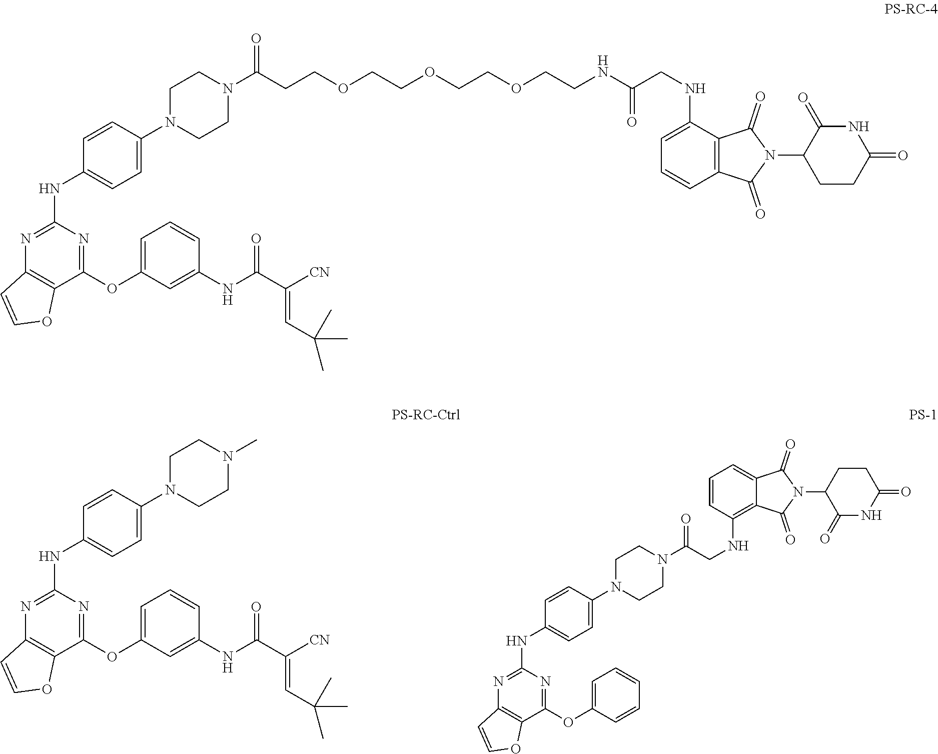 [SMILES: CC(C)(C)/C=C(\C#N)C(=O)Nc1cccc(Oc2nc(Nc3ccc(N4CCN(C(=O)CCOCCOCCOCCNC(=O)CNc5cccc6c5C(=O)N(C5CCC(=O)NC5=O)C6=O)CC4)cc3)nc3ccoc23)c1 ; CN1CCN(c2ccc(Nc3nc(Oc4cccc(NC(=O)/C(C#N)=C/C(C)(C)C)c4)c4occc4n3)cc2)CC1 ; O=C1CCC(N2C(=O)c3cccc(NCC(=O)N4CCN(c5ccc(Nc6nc(Oc7ccccc7)c7occc7n6)cc5)CC4)c3C2=O)C(=O)N1] 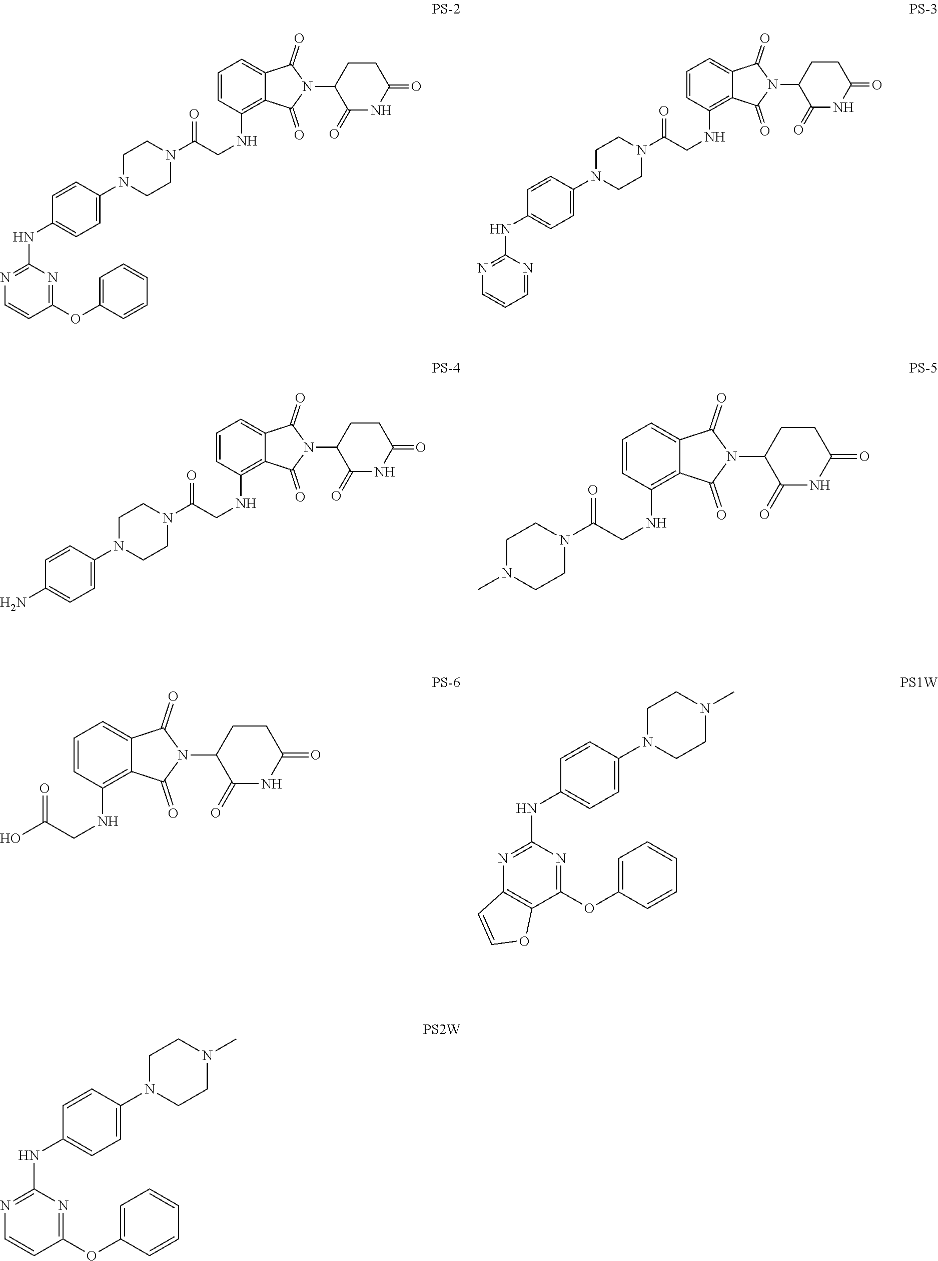
or a pharmaceutically acceptable salt or prodrug thereof.

2. A composition comprising a compound of claim 1 and a pharmaceutically acceptable carrier.

3. A kit comprising a compound of claim 1.

4. A method of treating or preventing a BTK-related disease in a subject, comprising:
administering to the subject an effective amount of a compound of claim 1.

5. The method of claim 4, wherein the BTK-related disease is cancer.

6. The method of claim 5, wherein the cancer is bladder cancer, blood cancer, a bone marrow cancer, brain cancer, breast cancer, bronchus cancer, colorectal cancer, cervical cancer, chondrosarcoma, endometrial cancer, gastrointestinal cancer, gastric cancer, genitourinary cancer, head and neck cancer, hepatic cancer, hepatocellular carcinoma, leukemia, liver cancer, lung cancer, lymphoma, melanoma of the skin, ovarian cancer, pancreatic cancer, prostate cancer, renal cancer, skin cancer, testicular cancer, thyroid cancer, or uterine cancer.

7. The method of claim 4, wherein the BTK-related disease is a neurodegenerative disorder.

8. The method of claim 4, wherein the BTK-related disease is an inflammatory disease.

9. The method of claim 4, further comprising administering a second compound, biomolecule, or composition.

10. The method of claim 9, wherein the second compound, biomolecule, or composition comprises a chemotherapeutic agent.

11. A method of inducing BTK degradation in a cell, comprising:
contacting a cell with an effective amount of a compound of claim 1.

12. The method of claim 11, wherein the contacting is performed in vitro.

13. The method of claim 11, wherein the contacting is performed in vivo.
